(19) United States
(12) Patent Application Publication
(10) Pub. No.: US 2026/0144980 A1
(43) Pub. Date: May 28, 2026

(54) MAGNETIC COUPLING FOR USE WITH A PERCUTANEOUS BLOOD PUMP

(71) Applicant: BOSTON SCIENTIFIC SCIMED, INC., Maple Grove, MN (US)

(72) Inventors: Travis J. Schauer, Rockford, MN (US); Benjamin Breidall, Eden Prairie, MN (US); Joseph Alan Kronstedt, New Hope, MN (US); Megan Johnson, Dayton, MN (US); Paul F. Chouinard, Maple Grove, MN (US)

(73) Assignee: BOSTON SCIENTIFIC SCIMED, INC., Maple Grove, MN (US)

(21) Appl. No.: 19/398,610

(22) Filed: Nov. 24, 2025

Related U.S. Application Data
(60) Provisional application No. 63/724,669, filed on Nov. 25, 2024.

(51) Int. Cl. A61M 60/419; A61M 60/122; A61M 60/216; A61M 60/416; A61M 60/538

(52) U.S. Cl. CPC A61M 60/419; A61M 60/122; A61M 60/216; A61M 60/416; A61M 60/538; A61M 2205/04; A61M 2205/103; A61M 2205/18; A61M 2205/3331; A61M 2205/3365; A61M 2205/50

(57) ABSTRACT

Disclosed are percutaneous circulatory support devices and related blood pump systems which may include a housing and an impeller within the housing. The impeller is adapted to rotate within the housing to move blood from a blood inlet to a blood outlet. A motor may be operably coupled to the impeller to rotate the impeller relative to the housing. A drive shaft may be coupled to the impeller to rotate with the impeller. A driving magnet assembly may be coupled to the motor and include a diametric dipole magnet having a single north pole and a single south pole. A driven magnet assembly, including a diametric dipole magnet, may be coupled to at least one of the drive shaft and the impeller, the driven magnet assembly magnetically coupled to the driving magnet assembly such that rotation of the driving magnet assembly causes rotation of the driven magnet assembly and the impeller.

### CROSS REFERENCE TP RELATED APPLICATIONS

The present application claims the benefit of U.S. Provisional Patent Application Ser. No. 63/724,669, filed Nov. 25, 2024, the disclosure of which is incorporated herein by reference.

### TECHNICAL FIELD

The present disclosure pertains to percutaneous blood pumps and related devices. More particularly, the present disclosure pertains to magnetic couplings for use with percutaneous circulatory support devices and the like.

### BACKGROUND

Percutaneous mechanical circulatory support devices, such as blood pumps can provide transient support in patients whose heart function or cardiac output is compromised. The percutaneous mechanical circulatory support devices may be sufficiently flexible to be navigated through the vasculature to a patient's heart. Such devices may be navigated through the aortic arch and placed across the aortic valve into the ventricle, for example. Various configurations of percutaneous mechanical circulatory support devices are known. However, there is an ongoing need to provide improved construction of percutaneous mechanical circulatory support devices.

### BRIEF SUMMARY

This disclosure provides design, material, manufacturing method, and use alternatives for medical devices, including percutaneous circulatory support devices and associated percutaneous blood pumps.

A first example is a percutaneous circulatory support device including a housing which includes a blood inlet and a blood outlet. An impeller may be disposed within the housing, the impeller configured and/or otherwise adapted to rotate relative to the housing to cause blood to flow through the housing from the blood inlet to the blood outlet. A motor may be operably coupled to the impeller and may be configured and/or otherwise adapted to rotate the impeller relative to the housing. An impeller shaft may be coupled to the impeller and configured and/or otherwise adapted to rotate with the impeller. A driving magnet assembly may be coupled to the motor, whereby the motor is configured and/or otherwise adapted to rotate the driving magnet assembly. A driven magnet assembly may be coupled to at least one of the impeller shaft and the impeller, the driven magnet may be magnetically coupled to the driving magnet assembly, whereby rotation of the driving magnet assembly causes rotation of the driven magnet assembly and the impeller. The driving magnet assembly may include a first dipole magnet having a single north pole and a single south pole, and the driven magnet assembly may include a second dipole magnet having a single north pole and a single south pole.

Alternatively or additionally to any of the examples described herein, the driving magnet assembly may apply a torque upon the driven magnet assembly during operation of the motor.

Alternatively or additionally to any of the examples described herein, the driving magnet assembly may apply a torque upon the driven magnet assembly during rotation of the impeller shaft.

Alternatively or additionally to any of the examples described herein, the percutaneous circulatory support device may include a yoke attached to one or more of the first dipole magnet and the second dipole magnet.

Alternatively or additionally to any of the examples described herein, the percutaneous circulatory support device may include a controller operably coupled thereto. The controller may be configured and/or otherwise adapted to automatically sense a magnetic decoupling event if an angle between the north pole of the first dipole magnet and the south pole of the second dipole magnet exceeds 90 degrees.

Alternatively or additionally to any of the examples described herein, the controller may be configured and/or otherwise adapted to issue an alarm or an alert that alerts the user of the percutaneous circulatory support device to the sensed magnetic decoupling event.

Alternatively or additionally to any of the examples described herein, the controller may be configured and/or otherwise adapted to automatically reduce the speed of the motor to permit magnetic recoupling between respective poles of the first dipole magnet and the second dipole magnet upon sensing a magnetic decoupling event.

Alternatively or additionally to any of the examples described herein, after automatically reducing the speed of the motor to permit magnetic recoupling, the controller may be configured to automatically increase the speed of the motor.

Alternatively or additionally to any of the examples described herein, the controller may be configured and/or otherwise adapted to permit magnetic recoupling between respective poles of the first dipole magnet and the second dipole magnet without user intervention.

Another example is a percutaneous blood pump including an impeller housing which includes an impeller, a motor housing including a motor, a first magnet assembly coupled to the motor such that the motor is configured to rotate the first magnet assembly, and a second magnet assembly coupled to the impeller and configured to rotate with the impeller. In this and other examples, the percutaneous blood pump may include a wall disposed between the first magnet assembly and the second magnet assembly. The first magnet assembly may be disposed proximal of the wall within the motor housing and remote from the impeller housing. The second magnet assembly may be disposed distal of the wall within the impeller housing and remote from the motor housing. The first magnet assembly may include a first diametric dipole magnet which may include a single north pole and a single south pole. The second magnet assembly may include a second diametric dipole magnet which may include a single north pole and a single south pole. The north pole of the first dipole magnet may be circumferentially aligned with the south pole of the second dipole magnet at rest.

Alternatively or additionally to any of the examples described herein, the first diametric dipole magnet may apply a torque upon the second diametric dipole magnet during operation of the motor.

Alternatively or additionally to any of the examples described herein, the north pole of the first diametric dipole magnet may be circumferentially offset from the south pole of the second diametric dipole magnet by an angle of 10 degrees to 85 degrees during normal operation of the motor.

Alternatively or additionally to any of the examples described herein, the first diametric dipole magnet and the second diametric dipole magnet may be separated by a distance within a range of about 0.5 mm to about 1.0 mm.

Alternatively or additionally to any of the examples described herein, the wall may allow the passage of magnetic forces between the first diametric dipole magnet and the second diametric dipole magnet.

Alternatively or additionally to any of the examples described herein, the percutaneous blood pump may include a controller operably connected thereto. The controller may be configured and/or otherwise adapted to automatically sense a magnetic decoupling event between the first diametric dipole magnet and the second diametric dipole magnet if the angle between the north pole of the first diametric dipole magnet and the south pole of the second diametric dipole magnet exceeds 90 degrees.

Alternatively or additionally to any of the examples described herein, the controller may automatically reduce the speed of the motor or stop the motor to permit magnetic recoupling between respective poles of the first diametric dipole magnet and the second diametric dipole magnet upon automatically sensing the magnetic decoupling event.

Alternatively or additionally to any of the examples described herein, after automatically reducing the speed of the motor to permit magnetic recoupling, the controller may be configured to automatically increase the speed of the motor.

Alternatively or additionally to any of the examples described herein, the controller may be configured and/or otherwise adapted to issue an alarm or an alert that alerts a user of the percutaneous blood pump to an automatically sensed magnet decoupling event.

Another example is a percutaneous circulatory support device. The device includes a controller, a housing comprising a blood inlet and a blood outlet, and an impeller disposed within the housing. The impeller is configured to rotate relative to the housing to cause blood to flow through the housing from the blood inlet to the blood outlet. The device includes a motor operably coupled to the impeller. The controller is configured to supply electrical current to the motor to rotate the impeller relative to the housing. A drive shaft is coupled to the impeller and configured to rotate with the impeller. A drive magnet is coupled to the motor. The motor is configured to rotate the drive magnet. A driven magnet is coupled to at least one of the drive shaft and the impeller. The driven magnet is magnetically coupled to the drive magnet, wherein rotation of the drive magnet causes rotation of the driven magnet and the impeller. The drive magnet is a dipole magnet having a single north pole and a single south pole, and the driven magnet is a dipole magnet having a single north pole and a single south pole.

Alternatively or additionally to any of the examples described herein, the controller is configured to automatically sense a magnetic decoupling event if an angle between the north pole of the driven magnet and the south pole of the driving magnet exceeds 90 degrees.

Alternatively or additionally to any of the examples described herein, the controller is configured to automatically reduce the speed of the motor to permit magnetic recoupling between respective poles of the driven magnet and the driving magnet upon automatically sensing the magnetic decoupling event.

Alternatively or additionally to any of the examples described herein, the controller is configured to permit magnetic recoupling between respective poles of the driven magnet and the drive magnet without user intervention.

Alternatively or additionally to any of the examples described herein, after automatically reducing the speed of the motor to permit magnetic recoupling, the controller may be configured to automatically increase the speed of the motor.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary percutaneous circulatory support device including a percutaneous blood pump;

FIG. 2 shows the distal end region of the percutaneous circulatory support device of FIG. 1 including the percutaneous blood pump;

FIG. 3 is a side view of a portion of the percutaneous blood pump of FIG. 1;

FIG. 4 is a cross-sectional view of the portion of the percutaneous blood pump of FIG. 3;

FIG. 5 is an enlarged view of an example magnetic coupling of the percutaneous circulatory support device of FIG. 4;

FIG. 6 shows a diagram of axial vs. diametric magnetization in accordance with the present disclosure;

FIGS. 7A-7D show a series of magnet assembly configurations;

FIGS. 8A-8C show a series of magnetic loading conditions with respect to operation of the devices disclosed herein;

FIG. 9 shows a chart of magnetic coupling results in accordance with examples described herein;

FIG. 10 shows a group of charts comparing time histories of motor speed against applied current in accordance with examples described herein; and

FIG. 11 shows a chart of motor speed during a magnetic recoupling event subsequent to a magnetic decoupling event.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

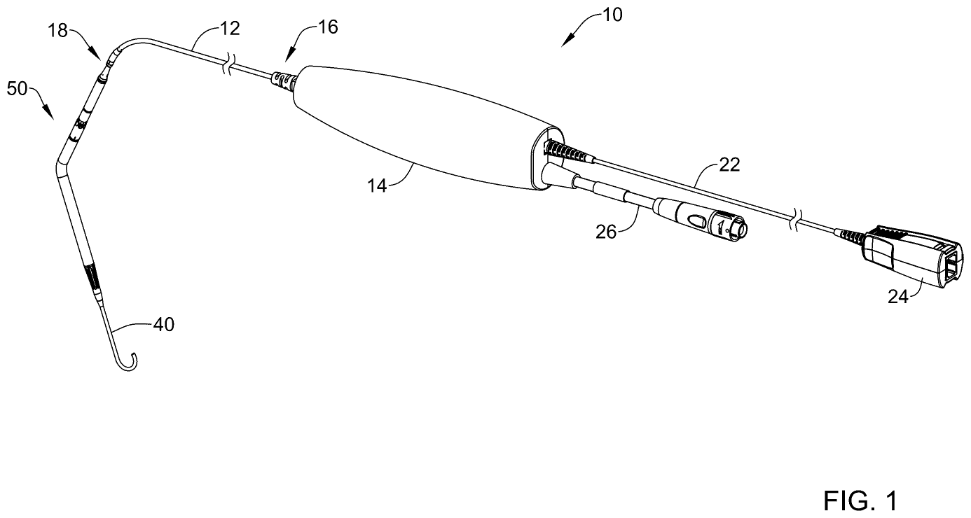

FIG. 1 illustrates a perspective view of a percutaneous circulatory support device 10 including a percutaneous blood pump 50 located at a distal end region thereof. The percutaneous circulatory support device 10 may be coupled to or include the blood pump 50, with an elongate shaft 12 of the percutaneous circulatory support device 10 extending proximally from the percutaneous blood pump 50 and a distal tip 40 extending distally from the blood pump 50. For instance, a proximal end 16 of the elongate shaft 12 may be coupled to a junction housing 14 and a distal end 18 of the elongate shaft 12 may be coupled to the percutaneous blood pump 50. An electrical cable 22 may extend from the junction housing 14 to a connector 24 at a proximal end thereof. The connector 24 may be configured to be connected to a controller (not shown) for controlling the blood pump 50, such as providing electrical power to the blood pump 50. The percutaneous circulatory support device 10 may also include an extension 26 connectable to the controller for sending and/or receiving signals, such as from one or more sensors during operation of the blood pump 50.

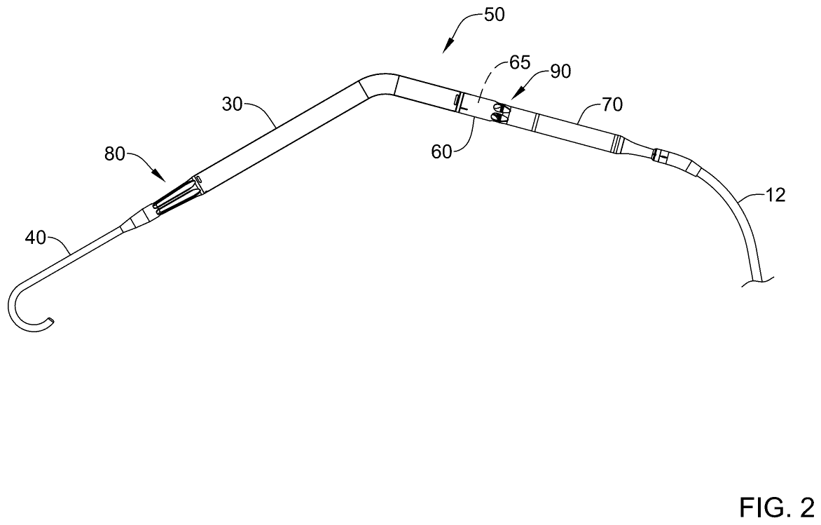

Additional features of the blood pump 50 are illustrated in FIG. 2 which shows the distal end region of the percutaneous circulatory support device of FIG. 1 including the percutaneous blood pump. The blood pump 50 may generally include a flexible cannula 30, an impeller housing 60, and a motor housing 70. In some embodiments, the flexible cannula 30, the impeller housing 60 and/or the motor housing 70 may be integrally or monolithically constructed. In other instances, the flexible cannula 30, the impeller housing 60 and/or the motor housing 70 may be separate components. The impeller housing 60 carries an impeller assembly 65 therein. The impeller assembly 65 may include an impeller, secured to an impeller shaft, that rotates relative to the impeller housing 60 to drive blood through the blood pump 50. In some embodiments, the impeller shaft and the impeller of the impeller assembly 65 may be integrally formed, whereas, in other embodiments the impeller shaft and the impeller may be separate components.

Rotation of the impeller causes blood to flow from a blood inlet 80 of the blood pump 50, such as at a distal end of the flexible cannula 30, through the flexible cannula 30 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the blood pump 50 (e.g., the flexible cannula 30). In some instances, the blood outlet 90 may include a plurality of blood outflow windows arranged around a circumference of the impeller housing 60. In other embodiments, the inlet 80 and/or the outlet 90 may be formed on, within, or proximate other portions of the blood pump 50.

With continued reference to FIG. 2, the motor housing 70 carries a motor configured to rotatably drive the impeller of the impeller assembly 65 relative to the impeller housing 60. Electrical power may be supplied to the motor through wiring extending through the elongate shaft 12, for example. In some instances, the motor may be physically connected to the impeller. For example, in some embodiments the impeller may be mounted on the drive shaft of the motor. In other embodiments, the impeller shaft may be directly or indirectly coupled to the drive shaft of the motor. In some instances, the drive assembly may include a magnetic coupling between the motor and the impeller. For example, a driving magnet may be mounted on the drive shaft of the motor. Rotation of the driving magnet causes rotation of a driven magnet, which is connected to the impeller assembly 65. More specifically, in embodiments incorporating an impeller shaft, the impeller shaft and the impeller of the impeller assembly 65 are configured to rotate with a driven magnet. In other embodiments, the motor may be coupled to the impeller assembly 65 via other components.

The blood pump 50 may be guided over a guidewire during introduction of the blood pump 50 into the vasculature of a patient. For instance, a guidewire, inserted through a guidewire lumen of the distal tip 40, may be advanced proximally along the impeller assembly 65 and out through one of the outflow windows of the blood outlet 90. With the guidewire tracked through the blood pump 50, the percutaneous circulatory support device 10 may be advanced over the guidewire into a vasculature.

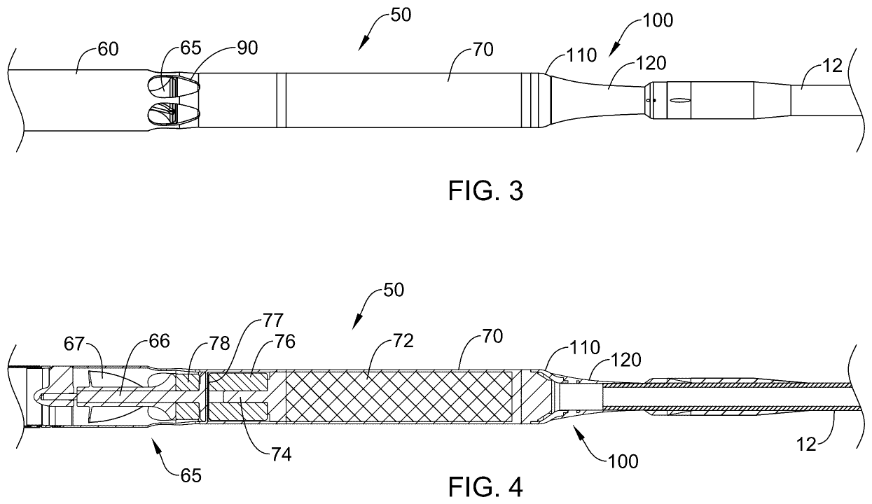

FIG. 3 is a side view of a portion of the percutaneous circulatory support device 10 illustrating a portion of the percutaneous blood pump 50 connected to the elongate shaft 12 at a junction 100. A proximal end of the impeller of the impeller assembly 65 is also visible through the outflow windows of the blood outlet 90. FIG. 4 is a cross-sectional view of the portion of the percutaneous circulatory support device 10 of FIG. 3, including the junction 100 between the percutaneous blood pump 50 and the elongate shaft 12 of the percutaneous circulatory support device 10. The junction 100 may be configured to mechanically connect the motor housing 70 (e.g., a metallic motor housing) of the blood pump 50, or other component of the blood pump 50, to the distal end region of the elongate shaft 12 (e.g., a polymeric tubular member).

The junction 100 may include an end cap 110 having a proximal end region surrounding the distal end region of the elongate shaft 12, and a fillet of material 120 surrounding the proximal end region of the end cap 110 and extending proximally therefrom. The fillet of material 120 may extend proximal of the end cap 110 and surround a portion of the elongate shaft 12 extending proximal of the end cap 110.

As shown in FIG. 4, the impeller assembly 65 includes an impeller shaft 66 and an impeller 67 coupled thereto, where the impeller shaft 66 is configured to rotate with the impeller 67. As shown, the impeller shaft 66 is at least partially disposed within the impeller 67. The impeller assembly 65 further includes a driven magnet 78 coupled to, and at least partially surrounding, the impeller shaft 66 and/or the impeller 67. The driven magnet 78 may be any type of magnetic rotor capable of being driven by a driving magnet assembly. In this manner, as a magnetic field is applied to the driven magnet 78 by the driving magnet assembly, the driven magnet 78 rotates, causing the impeller shaft 66 and impeller 67 to rotate.

Continuing with FIG. 4, the driving magnet assembly includes a drive magnet 76 coupled to a drive shaft 74 extending from a motor 72 configured to transfer torque from the motor 72 to the drive magnet 76. The motor housing 70 may be configured to hermetically seal the drive magnet 76 and the motor 72 within the motor housing 70, and thus fluidly isolate the drive magnet 76 from the driven magnet 78. A wall 77, may be disposed between the drive magnet 76 and the driven magnet 78. Wall 77 may be composed and/or comprised of any feasible material such that it allows magnetic forces (i.e., the extension and/or proliferation and/or propagation of a magnetic field) to pass through wall 77 and act upon magnets disposed on either side of wall 77, including but not limited to drive magnet 76 and driven magnet 78. In this and other examples, the relationship between (i.e., the combination of) drive magnet 76 and the driven magnet 78 may be referred to as a magnetic coupling. In other words, the driven magnet 78 of the impeller assembly 65 and the drive magnet 76 driven by rotation of the motor 72 may be magnetically coupled to form a magnetic coupling therebetween. This magnetic coupling conveys myriad advantages, including but not limited to, allowing the motor housing 70, including the motor 72, to be hermetically sealed from its surroundings while transmitting torque generated by the motor 72 to rotate the impeller 67. This eliminates the need for the use of motor purge fluid and further disallows blood from entering the motor housing 70, in other words preventing ingress of blood into the internal components and other components of the motor 72 so as to prevent motor failure.

In other non-limiting examples, the relationship between drive magnet 76, driven magnet 78 and wall 77 (i.e., the combination of) may be referred to as a magnetic coupling. In yet other non-limiting examples, the relationship between (i.e., the combination of) any magnetic elements and/or any ferromagnetic elements, and/or any elements that support the transfer and/or passage and/or propagation of magnetic force within the devices disclosed herein may be referred to as a magnetic coupling.

The drive magnet 76 may be a dipole magnet having a single north pole and a single south pole. For instance, in some instances the drive magnet 76 may be a cylindrical magnet having a north pole oriented to one semi-circular region of the cylindrical magnet (on a first radial side of the rotational axis of the drive magnet 76) and an opposing south pole oriented directly opposite the north pole in a second semi-circular region (on an opposite, second radial side of the rotational axis of the drive magnet 76) of the cylindrical magnet.

The driven magnet 78 may be a dipole magnet having a single north pole and a single south pole. For instance, in some instances the driven magnet 78 may be a cylindrical magnet having a south pole oriented to one semi-circular region of the cylindrical magnet (on a first radial side of the rotational axis of the driven magnet 78) and an opposing north pole oriented directly opposite the south pole in a second semi-circular region (on an opposite, second radial side of the rotational axis of the driven magnet 78) of the cylindrical magnet.

The north pole of the first dipole magnet (i.e., the drive magnet 76) may be attracted to the south pole of the second dipole magnet (i.e., the driven magnet 78) such that the north pole of the first dipole magnet (i.e., the drive magnet 76) is circumferentially aligned with the south pole of the second dipole magnet (i.e., the driven magnet 78) at rest. Furthermore, the south pole of the first dipole magnet (i.e., the drive magnet 76) may be attracted to the north pole of the second dipole magnet (i.e., the driven magnet 78) such that the south pole of the first dipole magnet (i.e., the drive magnet 76) is circumferentially aligned with the north pole of the second dipole magnet (i.e., the driven magnet 78) at rest.

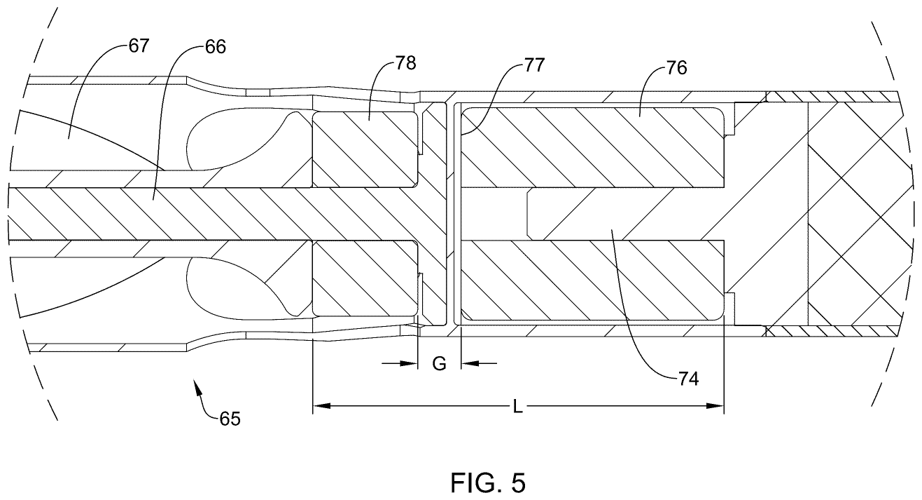

Turning to FIG. 5, an enlarged view of the magnetic coupling of FIG. 4 is shown. The wall 77 may separate drive magnet 76 from driven magnet 78 while allowing the passage, transfer, and/or proliferation and/or propagation of magnetic forces, including but not limited to electromagnetic forces, electromagnetic fields, magnetic forces, magnetic fields and/or any of the like and/or any combination of the aforementioned. Wall 77 may be formed of a ferromagnetic material, a magnetic material, a non-magnetic material, and/or a material conducive to the passage, transfer and/or proliferation and/or propagation of any of the magnetic and/or electromagnetic forces and/or fields described herein.

Continuing with FIG. 5, a gap distance G is shown which represents the distance of a desired gap between the drive magnet 76 and the driven magnet 78. The gap distance G is measured longitudinally from a distalmost extent (e.g., distal face) of the drive magnet 76 to a proximalmost extent (e.g., a proximal face) of the driven magnet 78. In a non-limiting sense, gap distance G may be dictated by a distance that may be dictated by the thickness of the wall 77, the extent of the impeller shaft 66, an air gap between the drive magnet 76 and the wall 77, and/or any bearings and/or other components residing between the drive magnet 76 and the driven magnet 78. In non-limiting examples, this distance (also known as a magnetic repulsion distance, a repulsion distance and gap distance G) may be within a range of about 0.1 millimeter (mm) to about 3 mm, and preferably less than 1 mm, such as in the range of about 0.5 mm to about 1 mm, or about 0.8 mm, dependent upon the magnetic repulsion forces emitted and/or elicited by the drive magnet 76 and/or the driven magnet 78 and/or any of the magnetic couplings and/or magnetic assemblies described herein.

Further shown in FIG. 5 is length distance L, which represents the overall length of the magnetic coupling assembly, which may be any combination of the illustrated and described parts within that distance as shown in FIG. 5 and applicable to other examples. In this and other examples, the overall length distance L may be any feasible distance that reduces an overall length (i.e., the length of the longest dimension of the magnetic coupling) such that it is advantageous to the user and patient of the device. The overall length L is measured longitudinally from a proximalmost extent (e.g., proximal face) of the drive magnet 76 to a distalmost extent (e.g., a distal face of the driven magnet 78. In some instances, the overall length L may be in the range of 1 mm to 10 mm, in the range of 1 mm to 5 mm, in the range of 3 mm to 8 mm, or in the range of 5 mm to 10 mm, for example.

Further shown in FIG. 5 is the interrelationship between the impeller assembly 65 and impeller shaft 66. As shown in FIG. 5 and applicable to other examples disclosed herein, the impeller 67 of the impeller assembly 65 may include one or more impeller blades and may be coupled to the impeller shaft 66 by any coupling means known in the art, including but not limited to an interference fit coupling, a snap-fit coupling, adhesive bond, and/or any other feasible coupling known in the art.

Driven magnet 78, as shown in FIG. 5 and applicable to other examples may be disposed along the impeller shaft 66 and may be positioned proximal of the impeller 67, however other configurations are contemplated, including but not limited to the driven magnet 78 being disposed distally of the impeller 67, for example. The impeller shaft 66 may extend proximally through the driven magnet 78. In some instances, the proximal end of the impeller shaft 66 may have an enlarged head such that the driven magnet 78 is positioned between the proximal end of the impeller 67 and the enlarged head of the impeller shaft 66. The enlarged head may act as a bearing surface for rotation of the impeller shaft 66.

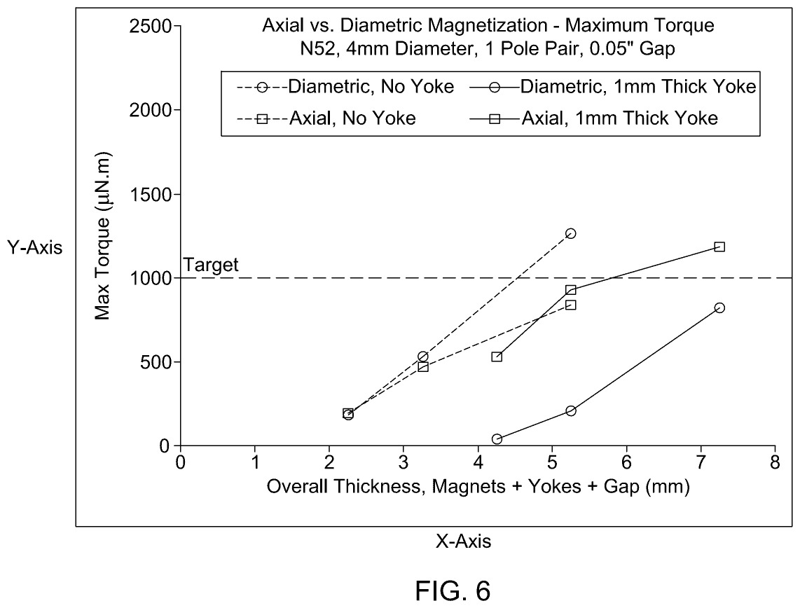

FIG. 6 shows a diagram of axial vs. diametric magnetization in accordance with examples described herein. The obtained and/or measured overall thickness of the magnetic couplings (distance L in FIG. 5), which includes axial thickness (h) of the magnets, gap distance (e) between the magnets, and yokes, if present, are plotted on the X-axis in increments of millimeters (mm). Max torque, measured and/or obtained and plotted in micro-Newton meters (μN·m) is charted along the Y-axis. As shown by the diagram in FIG. 6, Applicant has found that a diametric dipole magnet may be optimal in many instances. As can be appreciated by the diagram in FIG. 6, a diametric dipole magnet is able to withstand high amounts of torque with minimal thickness of the diametric dipole magnet assembly, which is advantageous as this allows for a reduced profile, size, and length of the magnetic coupling and the housings associated with the magnetic assemblies described herein. Use of a diametric dipole magnet also reduces the overall profile, size, and length of the percutaneous blood pumps and related devices disclosed herein. Applicant has found that diametric magnetization (e.g., the implementation of a dipole magnetic surface, and/or dipole magnet and/or diametric dipole magnet) as opposed to axial magnetization (e.g., the axial stacking of magnetic poles and/or magnets) provides better coupling under torque per unit of length, surface area, and volume occupied by a magnetic assembly. In other words, the reduced thickness and size afforded by the configuration and modeled by the dotted line referencing the diametric dipole magnet without a yoke, which is applicable to this and other examples, affords a smaller device for deployment into a patient with greater maximum torque, and therefore results in greater efficacy of the devices disclosed herein and greater reduction in cost for producing the devices disclosed herein while offering a greater level of safety to the patient using and/or subject to the devices disclosed herein.

Data supporting the axial magnetization configurations of the diagram of FIG. 6 is shown in Table 1, below. Table 1 provides data of an axial magnetization of drive and driven magnets of various overall length configurations, including various axial magnet thicknesses (i.e., each magnet has a thickness of 0.5 mm, 1.0 mm, 2.0 mm or 3 mm), with or without a yoke, and which are axially spaced apart by a gap of 0.025 inches or 0.05 inches.

TABLE 1

| | Magnet | Yoke | | | | | | | Total | Max |
|---|---|---|---|---|---|---|---|---|---|---|
| | Thickness, | Thickness, | Torque | | Axial Force | | | | Coupling | Axial |
| Gap, e | h | h_{yoke} | Max | Theta | Max− | Theta | Max+ | Theta | Length | Force |
| (inch) | (mm) | (mm) | (μN · m) | (deg) | (g) | (deg) | (g) | (deg) | (mm) | (g) |
| 0.025 | 0.5 | 0 | 427.1 | | −80.647 | | 79.555 | | 1.635 | 80.647 |
| 0.025 | 0.5 | 1 | 1102.9 | | −213.86 | | 143.74 | | 3.635 | 213.86 |
| 0.025 | 1 | 0 | 1013.7 | | −173.46 | | 165.25 | | 2.635 | 173.46 |
| 0.025 | 1 | 1 | 1867.4 | | −284.55 | | 247.98 | | 4.635 | 284.55 |
| 0.025 | 2 | 0 | 1753 | | −269.96 | | 250.72 | | 4.635 | 269.96 |
| 0.025 | 2 | 1 | 2322.3 | | −310.49 | | 307.12 | | 6.635 | 310.49 |
| 0.05 | 0.5 | 0 | 192.2 | | −28.876 | | 27.947 | | 2.27 | 28.876 |
| 0.05 | 0.5 | 1 | 525.12 | | −66.408 | | 65.958 | | 4.27 | 66.408 |
| 0.05 | 1 | 0 | 466.15 | | −63.939 | | 61.956 | | 3.27 | 63.939 |
| 0.05 | 1 | 1 | 921.63 | | −102.26 | | 113.51 | | 5.27 | 113.51 |
| 0.05 | 2 | 0 | 833.66 | | −108.02 | | 100.93 | | 5.27 | 108.02 |
| 0.05 | 2 | 1 | 1180.9 | | −120.26 | | 140.54 | | 7.27 | 140.54 |
| 0.05 | 3 | 1 | 1236 | | −120.26 | | 147.7 | | 9.27 | 147.7 |

Data supporting the diametric magnetization configurations of the diagram of FIG. 6 is shown in Table 2, below. Table 2 provides data of a diametric magnetization of drive and driven magnets of various overall length configurations, including various diametric magnet thicknesses (i.e., each magnet has a thickness of 0.5 mm, 1.0 mm, 2.0 mm or 3 mm), with or without a yoke, and which are axially spaced apart by a gap of 0.025 inches or 0.05 inches.

TABLE 2

| | Magnet | Yoke | | | | | | | Total | Max |
|---|---|---|---|---|---|---|---|---|---|---|
| | Thickness, | Thickness, | Torque | | Axial Force | | | | Coupling | Axial |
| Gap, e | h | h_{yoke} | Max | Theta | Max− | Theta | Max+ | Theta | Length | Force |
| (inch) | (mm) | (mm) | (μN · m) | (deg) | (g) | (deg) | (g) | (deg) | (mm) | (g) |
| 0.025 | 0.5 | 0 | 314 | 90, | −30.3 | 0 | 27.5 | 180 | 1.635 | 30.3 |
| | | | | 270 | | | | | | |
| 0.025 | 0.5 | 1 | 69 | 90, | −0.3 | 0 | 17.1 | 180 | 3.635 | 17.1 |
| | | | | 270 | | | | | | |
| 0.025 | 1 | 0 | 879 | 90, | −78.8 | 0 | 68.3 | 180 | 2.635 | 78.8 |
| | | | | 270 | | | | | | |
| 0.025 | 1 | 1 | 367 | 90, | −15.7 | 0 | 61.1 | 180 | 4.635 | 61.1 |
| | | | | 270 | | | | | | |
| 0.025 | 2 | 0 | 1970 | 90, | −152.5 | 0 | 139.1 | 180 | 4.635 | 152.5 |
| | | | | 270 | | | | | | |
| 0.025 | 2 | 1 | 1329 | 90, | −104.1 | 0 | 108.2 | 180 | 6.635 | 108.2 |
| | | | | 270 | | | | | | |
| 0.05 | 0.5 | 0 | 183 | 90, | −12.8 | 0 | 15.9 | 180 | 2.27 | 15.9 |
| | | | | 270 | | | | | | |
| 0.05 | 0.5 | 1 | 35 | 90, | 0 | — | 11 | 150 | 4.27 | 11 |
| | | | | 270 | | | | | | |
| 0.05 | 1 | 0 | 529 | 90, | −38.3 | 0 | 38.4 | 180 | 3.27 | 38.4 |
| | | | | 270 | | | | | | |
| 0.05 | 1 | 1 | 206 | 90, | −2.9 | 30 | 37.3 | 165 | 5.27 | 37.3 |
| | | | | 270 | | | | | | |
| 0.05 | 2 | 0 | 1260 | 90, | −83.4 | 0 | 82 | 180 | 5.27 | 83.4 |
| | | | | 270 | | | | | | |
| 0.05 | 2 | 1 | 818 | 90, | −46.5 | 0 | 68.9 | 180 | 7.27 | 68.9 |
| | | | | 270 | | | | | | |
| 0.05 | 3 | 1 | 1446 | 90, | −70.6 | 0 | 117.2 | 180 | 9.27 | 117.2 |
| | | | | 270 | | | | | | |

Additionally, data reproduced in Table 3 below displays the relationship between forces applied to and/or received by axial magnets in relation to gap distance and polar angle in axial magnets and/or axial magnetic assemblies including a 1 mm thick yoke and/or similar element. A yoke may be defined as a connector and/or a bar or frame or block connected to a magnet or magnetic assembly as will be described further herein.

TABLE 3
1 mm Yoke

| | | | | Axial | | | | Radial |
|---|---|---|---|---|---|---|---|---|
| gap | h_mag | theta | Torque | Force | x Force | y Force | \|Torque\| | Force |
| (inch) | (mm) | (deg) | (uN/m) (1) | (g) (1) | (g) (1) | (g) (1) | (uN/m) (1) | (g) |
| 0.025 | 0.5 | 0 | −1.4248 | −213.86 | −0.42814 | 0.055757 | 1.4248 | 0.4318 |
| 0.025 | 0.5 | 15 | −443.69 | −203.27 | 0.014203 | −0.11205 | 443.69 | 0.1129 |
| 0.025 | 0.5 | 30 | −751.98 | −176.3 | −0.37267 | −0.15552 | 751.98 | 0.4038 |
| 0.025 | 0.5 | 45 | −932.81 | −142.4 | −0.94048 | 0.74488 | 932.81 | 1.1997 |
| 0.025 | 0.5 | 60 | −1035.3 | −107.23 | 0.1752 | 0.20396 | 1035.3 | 0.2689 |
| 0.025 | 0.5 | 75 | −1088.4 | −71.954 | 0.51537 | 0.2218 | 1088.4 | 0.5611 |
| 0.025 | 0.5 | 90 | −1099.8 | −35.15 | −0.22211 | 0.068217 | 1099.8 | 0.2323 |
| 0.025 | 0.5 | 105 | −1078.6 | 1.6959 | 0.62584 | −0.00166 | 1078.6 | 0.6258 |
| 0.025 | 0.5 | 120 | −1032.5 | 37.44 | 0.64219 | 1.052 | 1032.5 | 1.2325 |
| 0.025 | 0.5 | 135 | −933.96 | 72.142 | −0.11834 | −0.11809 | 933.96 | 0.1672 |
| 0.025 | 0.5 | 150 | −751.83 | 105.84 | 0.49241 | 1.1199 | 751.83 | 1.2234 |
| 0.025 | 0.5 | 165 | −440.34 | 132.62 | −0.17369 | 0.46608 | 440.34 | 0.4974 |
| 0.025 | 0.5 | 180 | −3.7173 | 143.74 | −0.00486 | 0.44434 | 3.7173 | 0.4444 |
| 0.025 | 0.5 | 195 | 447.01 | 132.88 | −0.45791 | 0.29707 | 447.01 | 0.5458 |
| 0.025 | 0.5 | 210 | 755.29 | 106.38 | −0.28298 | 0.86443 | 755.29 | 0.9096 |
| 0.025 | 0.5 | 225 | 935.75 | 72.673 | −0.19093 | −0.67203 | 935.75 | 0.6986 |
| 0.025 | 0.5 | 240 | 1036.8 | 37.827 | −0.35833 | 0.078966 | 1036.8 | 0.3669 |
| 0.025 | 0.5 | 255 | 1084.3 | 1.9883 | 0.040826 | −0.42008 | 1084.3 | 0.4221 |
| 0.025 | 0.5 | 270 | 1102.9 | −34.29 | 0.64195 | 0.092592 | 1102.9 | 0.6486 |
| 0.025 | 0.5 | 285 | 1085.2 | −70.439 | 0.039938 | −0.36346 | 1085.2 | 0.3656 |
| 0.025 | 0.5 | 300 | 1038.7 | −108.04 | −0.36588 | −0.10092 | 1038.7 | 0.3795 |
| 0.025 | 0.5 | 315 | 937.47 | −143.86 | −0.13782 | 0.45071 | 937.47 | 0.4713 |
| 0.025 | 0.5 | 330 | 753.37 | −176.89 | 0.16123 | −0.1471 | 753.37 | 0.2183 |
| 0.025 | 0.5 | 345 | 442.73 | −203.17 | −0.26127 | −0.15823 | 442.73 | 0.3054 |
| 0.025 | 0.5 | 360 | 0.11707 | −213.69 | −0.00259 | −0.03432 | 0.11707 | 0.0344 |
| 0.025 | 1 | 0 | −0.30576 | −284.55 | 0.47748 | 0.2643 | 0.30576 | 0.5457 |
| 0.025 | 1 | 15 | −669.38 | −270.45 | −0.43269 | −0.22847 | 669.38 | 0.4893 |
| 0.025 | 1 | 30 | −1173.2 | −233.65 | 0.48279 | −0.70254 | 1173.2 | 0.8524 |
| 0.025 | 1 | 45 | −1509.1 | −184.51 | −0.46831 | −0.47493 | 1509.1 | 0.6670 |
| 0.025 | 1 | 60 | −1718.3 | −131.45 | 0.72399 | 0.25442 | 1718.3 | 0.7674 |
| 0.025 | 1 | 75 | −1824.7 | −74.88 | 0.63213 | 0.3751 | 1824.7 | 0.7350 |
| 0.025 | 1 | 90 | −1867.4 | −16.61 | 1.222 | 0.49335 | 1867.4 | 1.3178 |
| 0.025 | 1 | 105 | −1822.1 | 40.298 | −0.07528 | −0.08597 | 1822.1 | 0.1143 |
| 0.025 | 1 | 120 | −1716.3 | 95.592 | 0.23767 | −0.82129 | 1716.3 | 0.8550 |
| 0.025 | 1 | 135 | −1514.4 | 148.37 | −0.00843 | 0.13683 | 1514.4 | 0.1371 |
| 0.025 | 1 | 150 | −1176.8 | 195.12 | −0.20433 | −0.73811 | 1176.8 | 0.7659 |
| 0.025 | 1 | 165 | −670.15 | 231.58 | −0.13203 | −0.46273 | 670.15 | 0.4812 |
| 0.025 | 1 | 180 | 4.6727 | 247.98 | 0.95036 | −0.76471 | 4.6727 | 1.2198 |
| 0.025 | 1 | 195 | 667.47 | 232.13 | 0.030045 | −1.2931 | 667.47 | 1.2934 |
| 0.025 | 1 | 210 | 1174.9 | 195.39 | 0.020947 | −1.6387 | 1174.9 | 1.6388 |
| 0.025 | 1 | 225 | 1513.5 | 149.69 | 1.5449 | 0.43127 | 1513.5 | 1.6040 |
| 0.025 | 1 | 240 | 1716.9 | 93.627 | −0.75883 | −0.31642 | 1716.9 | 0.8222 |
| 0.025 | 1 | 255 | 1836.2 | 38.061 | 0.62913 | −0.22102 | 1836.2 | 0.6668 |
| 0.025 | 1 | 270 | 1862.2 | −17.874 | −0.35001 | 0.13802 | 1862.2 | 0.3762 |
| 0.025 | 1 | 285 | 1830.7 | −74.863 | −0.18421 | 0.10797 | 1830.7 | 0.2135 |
| 0.025 | 1 | 300 | 1717.7 | −131.42 | 0.23713 | −0.35955 | 1717.7 | 0.4307 |
| 0.025 | 1 | 315 | 1509.9 | −184.63 | −0.74836 | −0.01669 | 1509.9 | 0.7485 |
| 0.025 | 1 | 330 | 1177.7 | −233.56 | −0.07142 | 0.58362 | 1177.7 | 0.5880 |
| 0.025 | 1 | 345 | 669.21 | −269.54 | 0.41063 | 0.23756 | 669.21 | 0.4744 |
| 0.025 | 1 | 360 | −2.5781 | −284.49 | −0.07245 | −0.07105 | 2.5781 | 0.1015 |
| 0.025 | 2 | 0 | 3.219 | −310.49 | −0.07135 | −0.60505 | 3.219 | 0.6092 |
| 0.025 | 2 | 15 | −793.02 | −294.31 | −0.10079 | 0.32804 | 793.02 | 0.3432 |
| 0.025 | 2 | 30 | −1412.2 | −252.81 | 0.014106 | −0.03417 | 1412.2 | 0.0370 |
| 0.025 | 2 | 45 | −1842.9 | −197.81 | 0.13861 | −0.07072 | 1842.9 | 0.1556 |
| 0.025 | 2 | 60 | −2118.4 | −135.5 | −0.40451 | −0.49302 | 2118.4 | 0.6377 |
| 0.025 | 2 | 75 | −2267.1 | −67.574 | −0.37934 | 0.5242 | 2267.1 | 0.6471 |
| 0.025 | 2 | 90 | −2315 | 0.16955 | 0.33289 | −0.4468 | 2315 | 0.5572 |
| 0.025 | 2 | 105 | −2272.7 | 66.024 | −0.25684 | 0.25082 | 2272.7 | 0.3590 |
| 0.025 | 2 | 120 | −2112.6 | 133.49 | −0.48455 | 0.2786 | 2112.6 | 0.5589 |
| 0.025 | 2 | 135 | −1843.4 | 194.66 | −0.60675 | −0.09694 | 1843.4 | 0.6144 |
| 0.025 | 2 | 150 | −1403.2 | 250.32 | −0.16571 | −0.31841 | 1403.2 | 0.3589 |
| 0.025 | 2 | 165 | −785.91 | 291.13 | −0.14386 | −0.5123 | 785.91 | 0.5321 |
| 0.025 | 2 | 180 | −2.2017 | 307.12 | −0.31861 | 0.93458 | 2.2017 | 0.9874 |
| 0.025 | 2 | 195 | 796.39 | 291.59 | −0.20832 | −0.21596 | 796.39 | 0.3001 |
| 0.025 | 2 | 210 | 1407.8 | 249.66 | 0.031994 | −0.8583 | 1407.8 | 0.8589 |
| 0.025 | 2 | 225 | 1841.9 | 194.77 | 0.55709 | −0.54093 | 1841.9 | 0.7765 |
| 0.025 | 2 | 240 | 2120.6 | 132.14 | −0.58949 | 0.74721 | 2120.6 | 0.9517 |
| 0.025 | 2 | 255 | 2274.9 | 66.393 | −0.23527 | 0.26791 | 2274.9 | 0.3565 |
| 0.025 | 2 | 270 | 2322.3 | −1.8076 | −0.37753 | 0.26976 | 2322.3 | 0.4640 |
| 0.025 | 2 | 285 | 2271.8 | −69.005 | −0.59979 | −0.34686 | 2271.8 | 0.6929 |
| 0.025 | 2 | 300 | 2119 | −135.36 | 0.037339 | −0.22168 | 2119 | 0.2248 |
| 0.025 | 2 | 315 | 1843.5 | −197.52 | −0.50207 | −0.16381 | 1843.5 | 0.5281 |
| 0.025 | 2 | 330 | 1409.2 | −253 | 0.15276 | 0.33745 | 1409.2 | 0.3704 |
| 0.025 | 2 | 345 | 794.42 | −293.98 | 0.13043 | −0.03867 | 794.42 | 0.1360 |
| 0.025 | 2 | 360 | 3.3748 | −310.08 | 0.04058 | −0.50948 | 3.3748 | 0.5111 |
| 0.05 | 0.5 | 0 | 2.4383 | −66.408 | −0.24366 | 0.65891 | 2.4383 | 0.7025 |
| 0.05 | 0.5 | 15 | −168.72 | −62.344 | −0.84154 | −1.0272 | 168.72 | 1.3279 |
| 0.05 | 0.5 | 30 | −311.11 | −56.208 | 0.23875 | −0.52531 | 311.11 | 0.5770 |
| 0.05 | 0.5 | 45 | −402.76 | −43.05 | −0.08323 | −0.33149 | 402.76 | 0.3418 |
| 0.05 | 0.5 | 60 | −476.45 | −30.641 | 0.56876 | −0.55747 | 476.45 | 0.7964 |
| 0.05 | 0.5 | 75 | −508.53 | −16.028 | −1.356 | 0.025429 | 508.53 | 1.3562 |
| 0.05 | 0.5 | 90 | −525.09 | 0.51788 | 0.73204 | −0.63659 | 525.09 | 0.9701 |
| 0.05 | 0.5 | 105 | −507.59 | 13.492 | 0.38209 | 0.57218 | 507.59 | 0.6880 |
| 0.05 | 0.5 | 120 | −474.45 | 31.059 | 0.49211 | 0.072508 | 474.45 | 0.4974 |
| 0.05 | 0.5 | 135 | −411.98 | 44.55 | −0.75587 | 0.36973 | 411.98 | 0.8415 |
| 0.05 | 0.5 | 150 | −316.53 | 54.384 | 0.77056 | 1.0355 | 316.53 | 1.2907 |
| 0.05 | 0.5 | 165 | −165.74 | 61.36 | 0.68881 | −0.0899 | 165.74 | 0.6947 |
| 0.05 | 0.5 | 180 | 2.1901 | 65.958 | 0.45027 | 0.3617 | 2.1901 | 0.5776 |
| 0.05 | 0.5 | 195 | 176.36 | 63.75 | 0.31908 | −0.0549 | 176.36 | 0.3238 |
| 0.05 | 0.5 | 210 | 307.55 | 56.071 | 0.11179 | 0.067605 | 307.55 | 0.1306 |
| 0.05 | 0.5 | 225 | 414.37 | 43.575 | −0.20411 | 0.74864 | 414.37 | 0.7760 |
| 0.05 | 0.5 | 240 | 472.47 | 28.868 | −1.93 | 0.10354 | 472.47 | 1.9328 |
| 0.05 | 0.5 | 255 | 518.44 | 14.827 | −0.3159 | −0.38814 | 518.44 | 0.5004 |
| 0.05 | 0.5 | 270 | 525.12 | 1.5878 | −1.0777 | 0.35578 | 525.12 | 1.1349 |
| 0.05 | 0.5 | 285 | 508.43 | −15.911 | −0.28422 | 0.70529 | 508.43 | 0.7604 |
| 0.05 | 0.5 | 300 | 473.12 | −29.779 | −0.94371 | −0.25536 | 473.12 | 0.9776 |
| 0.05 | 0.5 | 315 | 404.31 | −44.933 | −0.57059 | 0.9302 | 404.31 | 1.0913 |
| 0.05 | 0.5 | 330 | 304.34 | −56.319 | −0.20735 | −0.03483 | 304.34 | 0.2103 |
| 0.05 | 0.5 | 345 | 167.89 | −65.227 | −0.52838 | 0.22126 | 167.89 | 0.5728 |
| 0.05 | 0.5 | 360 | 1.875 | −65.605 | 0.36485 | −0.33833 | 1.875 | 0.4976 |
| 0.05 | 1 | 0 | 5.9336 | −102.26 | −1.189 | −0.04926 | 5.9336 | 1.1900 |
| 0.05 | 1 | 15 | −282.28 | −98.675 | −0.20446 | 0.17634 | 282.28 | 0.2700 |
| 0.05 | 1 | 30 | −520 | −83.633 | 1.8267 | −2.3084 | 520 | 2.9437 |
| 0.05 | 1 | 45 | −697.71 | −64.544 | 1.3583 | 1.0366 | 697.71 | 1.7087 |
| 0.05 | 1 | 60 | −816.78 | −43.475 | 0.98004 | −0.01999 | 816.78 | 0.9802 |
| 0.05 | 1 | 75 | −893.72 | −20.129 | −0.11912 | −0.15985 | 893.72 | 0.1994 |
| 0.05 | 1 | 90 | −918.88 | 6.345 | 0.65916 | −0.49694 | 918.88 | 0.8255 |
| 0.05 | 1 | 105 | −890.46 | 30.666 | 0.90849 | 0.80228 | 890.46 | 1.2120 |
| 0.05 | 1 | 120 | −820.95 | 54.611 | 0.66893 | 0.45702 | 820.95 | 0.8101 |
| 0.05 | 1 | 135 | −698.86 | 77.105 | 1.2588 | −0.27223 | 698.86 | 1.2879 |
| 0.05 | 1 | 150 | −511.69 | 95.462 | 1.0768 | 0.27069 | 511.69 | 1.1103 |
| 0.05 | 1 | 165 | −280.65 | 108.07 | 1.2263 | 0.64178 | 280.65 | 1.3841 |
| 0.05 | 1 | 180 | −2.7037 | 113.51 | −0.30925 | 0.45697 | 2.7037 | 0.5518 |
| 0.05 | 1 | 195 | 263.9 | 109.18 | −0.16728 | 0.67886 | 263.9 | 0.6992 |
| 0.05 | 1 | 210 | 510.95 | 95.446 | 2.1928 | −0.88785 | 510.95 | 2.3657 |
| 0.05 | 1 | 225 | 698.08 | 76.716 | 0.088431 | 0.12642 | 698.08 | 0.1543 |
| 0.05 | 1 | 240 | 826.33 | 56.642 | 0.14761 | −0.22644 | 826.33 | 0.2703 |
| 0.05 | 1 | 255 | 894.12 | 30.076 | −0.25069 | −0.01086 | 894.12 | 0.2509 |
| 0.05 | 1 | 270 | 921.63 | 6.9277 | 0.60088 | 0.012793 | 921.63 | 0.6010 |
| 0.05 | 1 | 285 | 890.27 | −18.557 | 1.045 | 1.6707 | 890.27 | 1.9706 |
| 0.05 | 1 | 300 | 835.31 | −43.051 | −2.3976 | 1.5405 | 835.31 | 2.8498 |
| 0.05 | 1 | 315 | 697.38 | −65.53 | 1.1985 | −1.3967 | 697.38 | 1.8404 |
| 0.05 | 1 | 330 | 518.56 | −83.585 | −0.10083 | −0.46699 | 518.56 | 0.4778 |
| 0.05 | 1 | 345 | 279.38 | −96.454 | 1.4224 | 0.67574 | 279.38 | 1.5748 |
| 0.05 | 1 | 360 | 2.2216 | −99.061 | 0.5449 | 1.23 | 2.2216 | 1.3453 |
| 0.05 | 2 | 0 | −2.5739 | −118.15 | 0.16634 | −0.33662 | 2.5739 | 0.3755 |
| 0.05 | 2 | 15 | −340.72 | −111.98 | 0.001427 | −0.37342 | 340.72 | 0.3734 |
| 0.05 | 2 | 30 | −646.8 | −97.152 | 1.102 | −0.43717 | 646.8 | 1.1855 |
| 0.05 | 2 | 45 | −885.52 | −75.702 | 1.0193 | −0.22424 | 885.52 | 1.0437 |
| 0.05 | 2 | 60 | −1054.6 | −47.417 | −0.98157 | −0.1473 | 1054.6 | 0.9926 |
| 0.05 | 2 | 75 | −1147.6 | −18.484 | 0.55997 | −0.868 | 1147.6 | 1.0330 |
| 0.05 | 2 | 90 | −1171.7 | 12.043 | −0.38852 | −0.2114 | 1171.7 | 0.4423 |
| 0.05 | 2 | 105 | −1149.1 | 42.053 | 0.69927 | 0.13756 | 1149.1 | 0.7127 |
| 0.05 | 2 | 120 | −1056.3 | 71.766 | 0.74138 | −1.3795 | 1056.3 | 1.5661 |
| 0.05 | 2 | 135 | −882.49 | 99.051 | 1.5582 | −0.3874 | 882.49 | 1.6056 |
| 0.05 | 2 | 150 | −654.94 | 119.4 | 1.4406 | 0.2854 | 654.94 | 1.4686 |
| 0.05 | 2 | 165 | −343.22 | 136.45 | −0.74997 | −0.2559 | 343.22 | 0.7924 |
| 0.05 | 2 | 180 | −6.8157 | 140.54 | −1.585 | −0.2392 | 6.8157 | 1.6029 |
| 0.05 | 2 | 195 | 345.09 | 134.66 | 0.30014 | 0.045635 | 345.09 | 0.3036 |
| 0.05 | 2 | 210 | 652.45 | 122.86 | −0.13155 | −1.1601 | 652.45 | 1.1675 |
| 0.05 | 2 | 225 | 892.95 | 98.807 | −0.87003 | −0.39882 | 892.95 | 0.9571 |
| 0.05 | 2 | 240 | 1052.5 | 72.853 | −0.58936 | 2.1239 | 1052.5 | 2.2042 |
| 0.05 | 2 | 255 | 1141.5 | 42.448 | −1.1887 | −0.16597 | 1141.5 | 1.2002 |
| 0.05 | 2 | 270 | 1180.9 | 11.953 | −0.60403 | −1.4354 | 1180.9 | 1.5573 |
| 0.05 | 2 | 285 | 1149.2 | −18.581 | −0.55536 | −0.48665 | 1149.2 | 0.7384 |
| 0.05 | 2 | 300 | 1047.6 | −48.237 | −0.75937 | 1.4147 | 1047.6 | 1.6056 |
| 0.05 | 2 | 315 | 882.73 | −75.785 | 0.79607 | 0.34765 | 882.73 | 0.8687 |
| 0.05 | 2 | 330 | 649.55 | −97.353 | 0.037023 | −0.1825 | 649.55 | 0.1862 |
| 0.05 | 2 | 345 | 346.35 | −113.27 | −0.08355 | −0.32513 | 346.35 | 0.3357 |
| 0.05 | 2 | 360 | −6.0477 | −120.26 | −0.36429 | 2.2399 | 6.0477 | 2.2693 |

Further, data reproduced in Table 4 below displays the relationship between forces applied to and/or received by axial magnets in relation to gap distance and polar angle in magnet assemblies that do not include a yoke and/or are devoid of a yoke as described herein.

TABLE 4
No Yoke

| | | | | Axial | | | | Radial |
|---|---|---|---|---|---|---|---|---|
| gap | h_mag | theta | Torque | Force | x Force | y Force | \|Torque\| | Force |
| (inch) | (mm) | (deg) | (uN/m) (1) | (g) (1) | (g) (1) | (g) (1) | (uN/m) (1) | (g) |
| 0.025 | 0.5 | 0 | −0.87779 | −80.647 | −0.87075 | −0.25261 | 0.87779 | 0.9067 |
| 0.025 | 0.5 | 15 | −203.45 | −74.604 | 0.20227 | 0.74719 | 203.45 | 0.7741 |
| 0.025 | 0.5 | 30 | −325.43 | −59.643 | −0.29544 | −0.07148 | 325.43 | 0.3040 |
| 0.025 | 0.5 | 45 | −382.84 | −44.654 | −0.94874 | 1.0715 | 382.84 | 1.4312 |
| 0.025 | 0.5 | 60 | −410.99 | −29.513 | 0.092463 | 0.63241 | 410.99 | 0.6391 |
| 0.025 | 0.5 | 75 | −426.44 | −15.396 | 0.59661 | 0.30519 | 426.44 | 0.6701 |
| 0.025 | 0.5 | 90 | −422.78 | −0.77413 | 0.48528 | −0.20681 | 422.78 | 0.5275 |
| 0.025 | 0.5 | 105 | −412.21 | 14.249 | 0.14441 | −0.14673 | 412.21 | 0.2059 |
| 0.025 | 0.5 | 120 | −408.68 | 29.771 | 0.44045 | 0.68625 | 408.68 | 0.8154 |
| 0.025 | 0.5 | 135 | −383.83 | 44.298 | 0.74785 | −0.05422 | 383.83 | 0.7498 |
| 0.025 | 0.5 | 150 | −328.13 | 59.931 | 0.33566 | 0.72493 | 328.13 | 0.7989 |
| 0.025 | 0.5 | 165 | −199.78 | 74.075 | 0.12895 | 0.33174 | 199.78 | 0.3559 |
| 0.025 | 0.5 | 180 | −2.9246 | 79.555 | −0.85349 | 0.3223 | 2.9246 | 0.9123 |
| 0.025 | 0.5 | 195 | 211.83 | 74.487 | −0.44198 | 0.52762 | 211.83 | 0.6883 |
| 0.025 | 0.5 | 210 | 330.04 | 60.444 | −0.84659 | 0.8383 | 330.04 | 1.1914 |
| 0.025 | 0.5 | 225 | 386.1 | 44.556 | −0.05649 | −0.99919 | 386.1 | 1.0008 |
| 0.025 | 0.5 | 240 | 410.98 | 29.758 | 0.059031 | 0.47757 | 410.98 | 0.4812 |
| 0.025 | 0.5 | 255 | 420.92 | 15.286 | −0.11877 | −0.41414 | 420.92 | 0.4308 |
| 0.025 | 0.5 | 270 | 427.1 | 0.34853 | 0.26606 | 0.068098 | 427.1 | 0.2746 |
| 0.025 | 0.5 | 285 | 422.59 | −14.518 | −0.11363 | −0.32346 | 422.59 | 0.3428 |
| 0.025 | 0.5 | 300 | 414.33 | −30.108 | −0.96809 | −0.5026 | 414.33 | 1.0908 |
| 0.025 | 0.5 | 315 | 389.77 | −46.8 | 0.035998 | 0.51449 | 389.77 | 0.5157 |
| 0.025 | 0.5 | 330 | 329.42 | −61.031 | 0.17228 | −0.11077 | 329.42 | 0.2048 |
| 0.025 | 0.5 | 345 | 205.81 | −74.101 | −0.45264 | −0.07011 | 205.81 | 0.4580 |
| 0.025 | 0.5 | 360 | −0.85315 | −79.514 | −0.20755 | −0.87255 | 0.85315 | 0.8969 |
| 0.025 | 1 | 0 | 3.903 | −173.36 | −0.0624 | 0.80918 | 3.903 | 0.8116 |
| 0.025 | 1 | 15 | −414.71 | −163.6 | −0.76534 | 1.0397 | 414.71 | 1.2910 |
| 0.025 | 1 | 30 | −696.36 | −139.25 | 1.7992 | −1.556 | 696.36 | 2.3787 |
| 0.025 | 1 | 45 | −865.92 | −103.7 | −1.1921 | −0.71901 | 865.92 | 1.3921 |
| 0.025 | 1 | 60 | −957.17 | −73.23 | 1.3738 | 1.7971 | 957.17 | 2.2621 |
| 0.025 | 1 | 75 | −986.73 | −37.26 | −0.34757 | 0.14935 | 986.73 | 0.3783 |
| 0.025 | 1 | 90 | −1013.7 | −2.6704 | 0.69723 | −0.09111 | 1013.7 | 0.7032 |
| 0.025 | 1 | 105 | −991.79 | 30.4 | 0.61406 | −0.35825 | 991.79 | 0.7109 |
| 0.025 | 1 | 120 | −959.75 | 65.267 | 2.6034 | −0.45211 | 959.75 | 2.6424 |
| 0.025 | 1 | 135 | −867.81 | 97.983 | −0.96065 | −0.19666 | 867.81 | 0.9806 |
| 0.025 | 1 | 150 | −705.08 | 127.09 | 0.37968 | 0.97765 | 705.08 | 1.0488 |
| 0.025 | 1 | 165 | −420.33 | 152 | −0.1322 | −0.64403 | 420.33 | 0.6575 |
| 0.025 | 1 | 180 | 1.499 | 165.25 | 1.095 | −1.1465 | 1.499 | 1.5854 |
| 0.025 | 1 | 195 | 418.15 | 152.94 | 0.10742 | −1.8007 | 418.15 | 1.8039 |
| 0.025 | 1 | 210 | 701.12 | 127.33 | −0.90455 | −0.71147 | 701.12 | 1.1508 |
| 0.025 | 1 | 225 | 874.25 | 98.61 | 1.2213 | 0.7668 | 874.25 | 1.4421 |
| 0.025 | 1 | 240 | 954.62 | 63.099 | −0.77778 | 0.3351 | 954.62 | 0.8469 |
| 0.025 | 1 | 255 | 1004.6 | 28.764 | 0.23554 | −0.76176 | 1004.6 | 0.7973 |
| 0.025 | 1 | 270 | 1009.1 | −3.4418 | −1.3757 | −0.72942 | 1009.1 | 1.5571 |
| 0.025 | 1 | 285 | 1001.7 | −37.052 | −0.80196 | 0.20737 | 1001.7 | 0.8283 |
| 0.025 | 1 | 300 | 959.2 | −72.496 | −0.06534 | −1.5483 | 959.2 | 1.5497 |
| 0.025 | 1 | 315 | 864.29 | −104.77 | −1.612 | −0.80301 | 864.29 | 1.8009 |
| 0.025 | 1 | 330 | 702.86 | −138.1 | 0.34376 | 0.78051 | 702.86 | 0.8529 |
| 0.025 | 1 | 345 | 419.03 | −160.63 | −0.65208 | 0.14033 | 419.03 | 0.6670 |
| 0.025 | 1 | 360 | 0.56373 | −173.46 | −0.10036 | −0.55036 | 0.56373 | 0.5594 |
| 0.025 | 2 | 0 | 6.1351 | −268.92 | 0.63411 | 1.226 | 6.1351 | 1.3803 |
| 0.025 | 2 | 15 | −638.9 | −253.63 | −0.62302 | 0.87041 | 638.9 | 1.0704 |
| 0.025 | 2 | 30 | −1115.5 | −218.43 | −1.6549 | −1.1503 | 1115.5 | 2.0154 |
| 0.025 | 2 | 45 | −1430.4 | −171.38 | −1.1783 | −0.65115 | 1430.4 | 1.3462 |
| 0.025 | 2 | 60 | −1622.1 | −118.49 | −0.95258 | −0.45244 | 1622.1 | 1.0546 |
| 0.025 | 2 | 75 | −1719.5 | −61.845 | −0.04907 | 1.4485 | 1719.5 | 1.4493 |
| 0.025 | 2 | 90 | −1737 | −7.952 | 0.51231 | −0.07833 | 1737 | 0.5183 |
| 0.025 | 2 | 105 | −1716.2 | 46.591 | −1.494 | 1.0131 | 1716.2 | 1.8051 |
| 0.025 | 2 | 120 | −1614.3 | 101.5 | −1.961 | 0.044729 | 1614.3 | 1.9615 |
| 0.025 | 2 | 135 | −1431.4 | 152.54 | −4.07 | 0.14837 | 1431.4 | 4.0727 |
| 0.025 | 2 | 150 | −1115.1 | 200.36 | −0.82249 | −1.2161 | 1115.1 | 1.4681 |
| 0.025 | 2 | 165 | −635.42 | 237.75 | 0.067271 | −0.31198 | 635.42 | 0.3192 |
| 0.025 | 2 | 180 | −4.0921 | 250.72 | −0.23645 | 2.0077 | 4.0921 | 2.0216 |
| 0.025 | 2 | 195 | 643.01 | 238.53 | −1.2608 | −0.22689 | 643.01 | 1.2811 |
| 0.025 | 2 | 210 | 1122.4 | 199.88 | −0.2883 | 0.19657 | 1122.4 | 0.3489 |
| 0.025 | 2 | 225 | 1430.8 | 153.99 | 2.5962 | −1.0053 | 1430.8 | 2.7840 |
| 0.025 | 2 | 240 | 1624.6 | 100.98 | −1.2529 | 2.2115 | 1624.6 | 2.5417 |
| 0.025 | 2 | 255 | 1731 | 47.16 | 0.31968 | 0.8063 | 1731 | 0.8674 |
| 0.025 | 2 | 270 | 1753 | −10.039 | −0.78284 | −0.53397 | 1753 | 0.9476 |
| 0.025 | 2 | 285 | 1719.6 | −63.738 | −0.40738 | −0.23757 | 1719.6 | 0.4716 |
| 0.025 | 2 | 300 | 1629.7 | −119 | 1.117 | 0.11462 | 1629.7 | 1.1229 |
| 0.025 | 2 | 315 | 1435.7 | −171.05 | −1.4224 | −0.40018 | 1435.7 | 1.4776 |
| 0.025 | 2 | 330 | 1109.7 | −218.28 | 1.0109 | −0.11459 | 1109.7 | 1.0174 |
| 0.025 | 2 | 345 | 638.99 | −253.66 | −0.76876 | −0.54849 | 638.99 | 0.9444 |
| 0.025 | 2 | 360 | 1.9597 | −269.96 | −0.21403 | −1.6382 | 1.9597 | 1.6521 |
| 0.05 | 0.5 | 0 | 1.3786 | −28.131 | −0.23812 | 1.1976 | 1.3786 | 1.2210 |
| 0.05 | 0.5 | 15 | −66.343 | −25.407 | −0.6775 | −0.8 | 66.343 | 1.0483 |
| 0.05 | 0.5 | 30 | −118.04 | −24.309 | 0.98541 | −0.2312 | 118.04 | 1.0122 |
| 0.05 | 0.5 | 45 | −145.88 | −17.339 | −0.60167 | −0.27492 | 145.88 | 0.6615 |
| 0.05 | 0.5 | 60 | −176.62 | −12.773 | −0.04791 | −0.49848 | 176.62 | 0.5008 |
| 0.05 | 0.5 | 75 | −181.08 | −7.1362 | −1.1491 | −0.09937 | 181.08 | 1.1534 |
| 0.05 | 0.5 | 90 | −189.69 | 0.28498 | −0.04466 | −0.42617 | 189.69 | 0.4285 |
| 0.05 | 0.5 | 105 | −181.24 | 4.143 | 0.35607 | 0.99793 | 181.24 | 1.0596 |
| 0.05 | 0.5 | 120 | −171.8 | 12.917 | −0.39967 | −0.77762 | 171.8 | 0.8743 |
| 0.05 | 0.5 | 135 | −155.71 | 18.26 | −0.92582 | 0.58731 | 155.71 | 1.0964 |
| 0.05 | 0.5 | 150 | −123.76 | 22.031 | 0.37353 | 0.77663 | 123.76 | 0.8618 |
| 0.05 | 0.5 | 165 | −66.717 | 25.822 | 0.36637 | 1.4784 | 66.717 | 1.5231 |
| 0.05 | 0.5 | 180 | 2.2095 | 27.947 | 0.25522 | 0.3384 | 2.2095 | 0.4239 |
| 0.05 | 0.5 | 195 | 72.437 | 27.017 | 0.38088 | 0.069907 | 72.437 | 0.3872 |
| 0.05 | 0.5 | 210 | 121.24 | 23.375 | −0.00718 | −0.2102 | 121.24 | 0.2103 |
| 0.05 | 0.5 | 225 | 158.61 | 18.722 | −0.34126 | 0.28563 | 158.61 | 0.4450 |
| 0.05 | 0.5 | 240 | 170.98 | 11.511 | −2.306 | −0.33211 | 170.98 | 2.3298 |
| 0.05 | 0.5 | 255 | 192.2 | 6.7875 | −0.08357 | −1.0787 | 192.2 | 1.0819 |
| 0.05 | 0.5 | 270 | 190.15 | 2.0192 | −0.53451 | 0.098029 | 190.15 | 0.5434 |
| 0.05 | 0.5 | 285 | 185.01 | −7.0141 | −0.48233 | −0.47477 | 185.01 | 0.6768 |
| 0.05 | 0.5 | 300 | 173.12 | −11.6 | −1.4496 | −0.1661 | 173.12 | 1.4591 |
| 0.05 | 0.5 | 315 | 145.89 | −19.337 | −0.58029 | 0.98544 | 145.89 | 1.1436 |
| 0.05 | 0.5 | 330 | 115.76 | −23.467 | 0.26395 | 0.49416 | 115.76 | 0.5602 |
| 0.05 | 0.5 | 345 | 67.214 | −28.876 | −0.77265 | 0.4188 | 67.214 | 0.8789 |
| 0.05 | 0.5 | 360 | −2.6321 | −26.502 | −0.16985 | −0.95678 | 2.6321 | 0.9717 |
| 0.05 | 1 | 0 | 4.5334 | −63.939 | −1.6674 | 0.7013 | 4.5334 | 1.8089 |
| 0.05 | 1 | 15 | −149.23 | −63.305 | 0.075039 | 1.3958 | 149.23 | 1.3978 |
| 0.05 | 1 | 30 | −268.9 | −53.377 | 2.1672 | −2.7103 | 268.9 | 3.4702 |
| 0.05 | 1 | 45 | −357.7 | −41.296 | 1.4019 | 1.3466 | 357.7 | 1.9439 |
| 0.05 | 1 | 60 | −418.81 | −29.345 | 1.3136 | 0.93312 | 418.81 | 1.6113 |
| 0.05 | 1 | 75 | −444.51 | −15.818 | −0.58484 | 0.44423 | 444.51 | 0.7344 |
| 0.05 | 1 | 90 | −456.45 | −1.5406 | −0.95383 | −0.16122 | 456.45 | 0.9674 |
| 0.05 | 1 | 105 | −444.89 | 12.169 | 1.6031 | 0.30183 | 444.89 | 1.6313 |
| 0.05 | 1 | 120 | −422.29 | 25.498 | 0.1153 | 1.2709 | 422.29 | 1.2761 |
| 0.05 | 1 | 135 | −362.19 | 39.684 | 1.1705 | −0.21219 | 362.19 | 1.1896 |
| 0.05 | 1 | 150 | −269.84 | 51.255 | 1.1928 | 1.2981 | 269.84 | 1.7629 |
| 0.05 | 1 | 165 | −154.92 | 58.263 | 0.38649 | 0.25781 | 154.92 | 0.4646 |
| 0.05 | 1 | 180 | −2.7226 | 61.956 | −0.87009 | 1.5284 | 2.7226 | 1.7587 |
| 0.05 | 1 | 195 | 149.97 | 60.129 | 0.28753 | 1.4969 | 149.97 | 1.5243 |
| 0.05 | 1 | 210 | 268.13 | 50.87 | 3.3887 | −1.4226 | 268.13 | 3.6752 |
| 0.05 | 1 | 225 | 366.67 | 39.031 | −0.02769 | 0.20423 | 366.67 | 0.2061 |
| 0.05 | 1 | 240 | 421.97 | 28.763 | 0.44533 | −0.54622 | 421.97 | 0.7048 |
| 0.05 | 1 | 255 | 446.69 | 11.939 | −0.85011 | −0.67558 | 446.69 | 1.0859 |
| 0.05 | 1 | 270 | 466.15 | 0.99259 | 2.3265 | 1.1064 | 466.15 | 2.5762 |
| 0.05 | 1 | 285 | 454.65 | −15.218 | 1.6715 | 1.3426 | 454.65 | 2.1439 |
| 0.05 | 1 | 300 | 420.65 | −29.371 | −4.4904 | 1.2437 | 420.65 | 4.6595 |
| 0.05 | 1 | 315 | 365.8 | −43.089 | −0.32996 | −2.6974 | 365.8 | 2.7175 |
| 0.05 | 1 | 330 | 272.35 | −54.349 | 0.30801 | −1.9921 | 272.35 | 2.0158 |
| 0.05 | 1 | 345 | 153.87 | −62.618 | 2.598 | 0.24501 | 153.87 | 2.6095 |
| 0.05 | 1 | 360 | 2.2465 | −62.92 | 0.34194 | 0.84782 | 2.2465 | 0.9142 |
| 0.05 | 2 | 0 | −1.0141 | −106.92 | 0.9745 | −1.3631 | 1.0141 | 1.6756 |
| 0.05 | 2 | 15 | −245.15 | −100.66 | 0.74846 | −0.67866 | 245.15 | 1.0103 |
| 0.05 | 2 | 30 | −469.31 | −88.948 | 1.1924 | −0.56723 | 469.31 | 1.3204 |
| 0.05 | 2 | 45 | −643.73 | −70.669 | 2.4491 | −0.56348 | 643.73 | 2.5131 |
| 0.05 | 2 | 60 | −752.25 | −48.183 | −0.61895 | 0.72987 | 752.25 | 0.9570 |
| 0.05 | 2 | 75 | −811.2 | −24.773 | −0.35657 | 0.63359 | 811.2 | 0.7270 |
| 0.05 | 2 | 90 | −826.92 | −1.563 | 1.0109 | −1.3929 | 826.92 | 1.7211 |
| 0.05 | 2 | 105 | −806.06 | 21.156 | 1.3961 | 1.871 | 806.06 | 2.3345 |
| 0.05 | 2 | 120 | −755.34 | 44.445 | 1.9909 | −0.77125 | 755.34 | 2.1351 |
| 0.05 | 2 | 135 | −632.09 | 66.889 | 0.73019 | −0.52498 | 632.09 | 0.8993 |
| 0.05 | 2 | 150 | −478.11 | 83.391 | 1.638 | −0.01815 | 478.11 | 1.6381 |
| 0.05 | 2 | 165 | −257.34 | 96.912 | −0.06566 | −0.54749 | 257.34 | 0.5514 |
| 0.05 | 2 | 180 | −2.3098 | 100.93 | −0.86846 | −1.283 | 2.3098 | 1.5493 |
| 0.05 | 2 | 195 | 258.98 | 96.119 | 0.92884 | −1.484 | 258.98 | 1.7507 |
| 0.05 | 2 | 210 | 478.53 | 86.07 | −0.59528 | −0.40851 | 478.53 | 0.7220 |
| 0.05 | 2 | 225 | 648.36 | 66.78 | 0.55331 | −0.55365 | 648.36 | 0.7827 |
| 0.05 | 2 | 240 | 754.32 | 46.223 | −0.30717 | 2.5622 | 754.32 | 2.5805 |
| 0.05 | 2 | 255 | 808.69 | 23.622 | −2.2108 | 0.4626 | 808.69 | 2.2587 |
| 0.05 | 2 | 270 | 833.66 | −3.9391 | 0.24036 | −3.231 | 833.66 | 3.2399 |
| 0.05 | 2 | 285 | 814.28 | −24.449 | −1.4691 | 0.48263 | 814.28 | 1.5463 |
| 0.05 | 2 | 300 | 741.86 | −50.088 | −1.8309 | 2.4628 | 741.86 | 3.0688 |
| 0.05 | 2 | 315 | 643.58 | −70.562 | −0.24436 | 0.5352 | 643.58 | 0.5883 |
| 0.05 | 2 | 330 | 482.7 | −88.735 | 0.072791 | 0.84328 | 482.7 | 0.8464 |
| 0.05 | 2 | 345 | 248 | −100.75 | −0.85617 | −1.4505 | 248 | 1.6843 |
| 0.05 | 2 | 360 | −8.5493 | −108.02 | −1.0299 | 4.9409 | 8.5493 | 5.0471 |

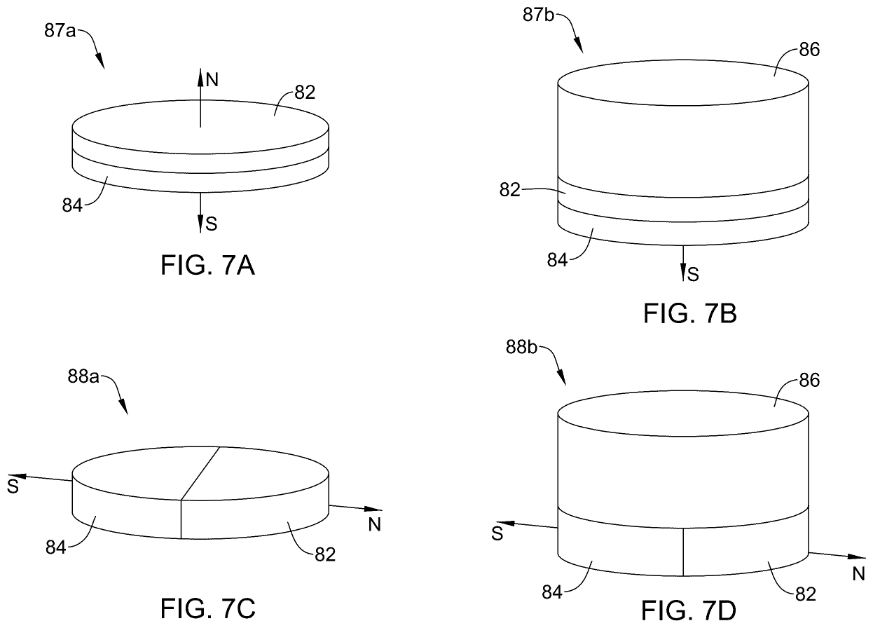

An axial magnet assembly 87a (which may also be referred to as an axial magnet) is shown in FIG. 7A. The axial magnet assembly 87a is representative of the “Axial, No Yoke” type in FIG. 6. In this and other examples, axial magnet assembly 87a may include a north pole 82 and a south pole 84 axially displaced from the north pole 82. The axial magnet assembly 87a is without or devoid of a yoke. A yoke 86 may be attached and/or otherwise included with the axial magnet assembly 87b as shown in FIG. 7B. The axial magnet assembly 87b is representative of the “Axial, 1 mm Thick Yoke” type in FIG. 6. Yoke 86 may ascribe to myriad thicknesses, widths and lengths; including but not limited to a thickness of about 1 mm or more, a length of about 1 mm to 10 mm or more, and a width of about 1 to about 10 mm or more.

FIG. 7C shows another example, in which a diametric magnet assembly 88a is provided with a north pole 82 and a south pole 84 diametrically opposite a rotational axis of the diametric magnet assembly 88a. The diametric magnet assembly 88a is without or devoid of a yoke. The axial magnet assembly 88a is representative of the “Diametric, No Yoke” type in FIG. 6. As shown in the above Tables, a diametric magnet assembly as described herein may provide optimal tolerance to torque and/or other forces applied to the diametric magnet assembly (i.e., axial force, radial force, normal force, etc.) Further, by providing a diametric magnet assembly devoid of a yoke, space may be conserved regarding the associated elements and devices disclosed herein. In other words, the reduced thickness and size afforded by the configuration of a diametric dipole magnet and/or a diametric magnet and/or a diametric magnet assembly, which is applicable to this and other examples, affords a smaller device for deployment into a patient, and therefore results in greater efficacy of the devices disclosed herein and greater reduction in cost for producing the devices disclosed herein while offering a greater level of safety to the patient using and/or subject by the devices disclosed herein. Further, and as an example as shown in FIG. 7D, a yoke 86 as described herein may be attached to and/or coupled to and/or otherwise included with a diametric magnet assembly 88b and/or dipole magnet assembly as disclosed herein. The diametric magnet assembly 88b is representative of the “Diametric, 1 mm Thick Yoke” type in FIG. 6. Yoke 86 may ascribe to myriad thicknesses, widths and lengths; including but not limited to a thickness of about 1 mm or more, a length of about 1 mm to 10 mm or more, and a width of about 1 to about 10 mm or more.

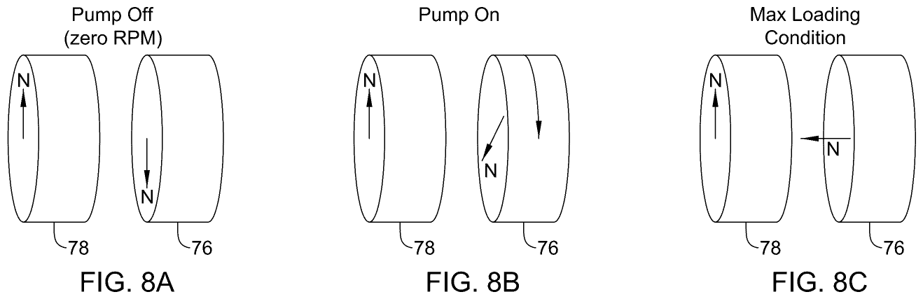

FIGS. 8A-8C show a series of magnetic loading conditions with respect to operation of the devices disclosed herein. FIG. 8A demonstrates a given polar angle between two diametrically magnetized dipole magnets, in a “Pump Off” condition in which both the first dipole magnet (e.g., the drive magnet 76) and the second dipole magnet (e.g., the driven magnet 78) are not rotating (i.e., zero RPMs or at rest), and therefore zero torque is applied from the first dipole magnet to the second dipole magnet. In other words, and in a non-limiting sense, since the motor is off (and thus the drive magnet is not rotating), the pump is off and not pumping blood. Thus, the driving mechanisms (such as the motor 72 and drive shaft 74 described herein) which, in the “Pump Off” condition, supply no rotation to the first dipole magnet (e.g., the drive magnet 76) and therefore supply zero RPMs (revolutions per minute) and zero torque from the first dipole magnet to the second dipole magnet, with the north pole of the first dipole magnet circumferentially aligned with the south pole of the second dipole magnet at rest. In other words, in the “Pump Off” condition, the opposite poles of the first and second dipole magnets are circumferentially aligned and no torque is applied therebetween.

FIG. 8B demonstrates a “Pump On” condition in which the first dipole magnet (e.g., drive magnet 76) is rotated by a drive shaft (such as drive shaft 74 extending from the motor 72) being rotated by the motor 72 such that the opposing poles of each dipole magnet become misaligned, therefore allowing the drive magnet (e.g., drive magnet 76) to impart torque and/or other angular forces and/or magnetic forces and/or electromagnetic forces upon the driven magnet (e.g., driven magnet 78). Thus, the magnetic fields between the first and second dipole magnets causes the second dipole magnet (e.g., the driven magnet 78) to rotate as the motor rotates the first dipole magnet (e.g., the drive magnet 76), with the north pole of the first dipole magnet misaligned from the south pole of the second dipole magnet (i.e., the north pole of the first dipole magnet is less than 180 degrees from the north pole of the second dipole magnet), generating torque to rotate the impeller.

FIG. 8C demonstrates a “Max Loading” condition in which the respective north poles of each dipole magnet (the north pole of the first dipole magnet and the north pole of the second dipole magnet) are orthogonal to one another. Said differently, the “Max Loading” condition is when the north pole of the first dipole magnet is misaligned or rotationally offset from the south pole of the second dipole magnet by 90 degrees. In other words, the “Max Loading” condition may be a scenario and/or condition where maximum torque is achieved and is imparted from the first dipole magnet (e.g., drive magnet 76) to the second magnet (e.g., driven magnet 78), and is achieved when the respective poles (e.g., the single north pole of one dipole magnet and the single north pole of another dipole magnet) of the drive magnet and the driven magnet reach an angle of 90 degrees with respect to one another. Angular misalignment beyond this “Max Loading” condition (e.g., rotationally misaligning the north pole of the first dipole magnet less than 90 degrees from the north pole of the second dipole magnet through increased torque applied by the motor) will cause magnetic decoupling between the first dipole magnet (e.g., the drive magnet 76) and the second dipole magnet (e.g., the driven magnet 78) such that the second dipole magnet is no longer being rotationally driven by the first dipole magnet, and thus will stop rotating (stopping rotation of the impeller).

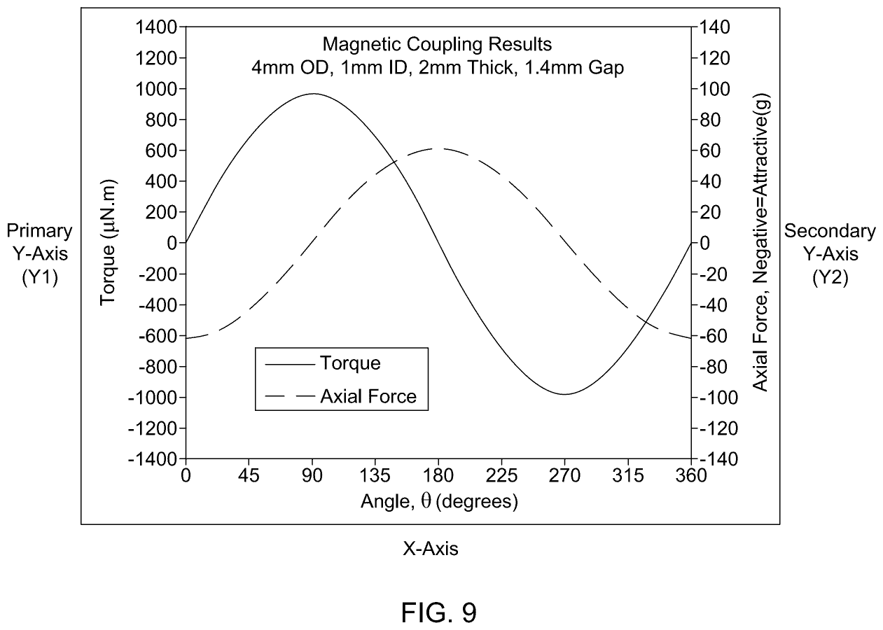

FIG. 9 shows a chart of magnetic coupling results in accordance with examples described herein. Plotted on the X-axis is the angle (θ) in degrees, and this value is plotted against torque (μN·m) on the Primary Y-axis (Y1) and axial force, in terms of net negative attractive forces on the Secondary Y-axis (Y2).

In the “Pump Off” condition, the magnetization directions of the dipole magnets are parallel to one another. In other words, the north poles of the magnets either possess the same magnetization direction and/or their fields and/or poles are pointing in the same direction or in exact opposite directions. When the pump is turned off (i.e., the motor is not energized), the magnetization directions are 180 degrees apart, which is commonly referred to as an angle of zero as shown in the plot of FIG. 9. In this instance, there exists a maximum attractive force (e.g., maximum attractive magnetic force, maximum magnetic attraction) between the drive magnet 76 and the driven magnet 78. When the magnetization directions of each magnet point in the exact same direction, or at an angle of 180 degrees, there exists a maximum repulsive force (e.g., maximum repulsive magnetic force, maximum magnetic repulsion) between the drive magnet 76 and the driven magnet 78. Between these two extremes (i.e., between these two conditions and/or scenarios) the torque rises from a value of zero and falls back to a value of zero, resulting in the sinusoidal shape of the curves shown in FIG. 9. The magnetization directions are 180 degrees apart when the pump is off due to the statically stable zero torque position, as shown in FIG. 9.

Data reproduced in Table 5 below displays angular results from the use of diametric magnetization that is provided in the Chart of FIG. 9. More particularly, the data reproduced in Table 5 displays the relationship between polar angle between the drive and driven diametric magnets to torque and axial force applied upon the diametric magnets and diametric magnet assemblies disclosed herein.

TABLE 5

| Angular Position | Torque | Axial Force |
|---|---|---|
| (degrees) | (μN · m) | (gram) |
| 0 | 4.5024 | −61.806 |
| 15 | 252.89 | −59.819 |
| 30 | 483.69 | −53.538 |
| 45 | 681.88 | −43.419 |
| 60 | 834.58 | −30.222 |
| 75 | 931.74 | −14.942 |
| 90 | 966.73 | 1.2875 |
| 105 | 936.81 | 17.293 |
| 120 | 843.39 | 31.962 |
| 135 | 692.11 | 44.323 |
| 150 | 492.67 | 53.605 |
| 165 | 258.31 | 59.271 |
| 180 | 4.9906 | 61.026 |
| 195 | −249.66 | 58.818 |
| 210 | −487.65 | 52.82 |
| 225 | −692.05 | 43.412 |
| 240 | −848.31 | 31.164 |
| 255 | −945.42 | 16.817 |
| 270 | −976.77 | 1.2538 |
| 285 | −940.6 | −14.53 |
| 300 | −839.98 | −29.482 |
| 315 | −682.5 | −42.555 |
| 330 | −479.5 | −52.786 |
| 345 | −245.18 | −59.384 |
| 360 | 4.5024 | −61.806 |

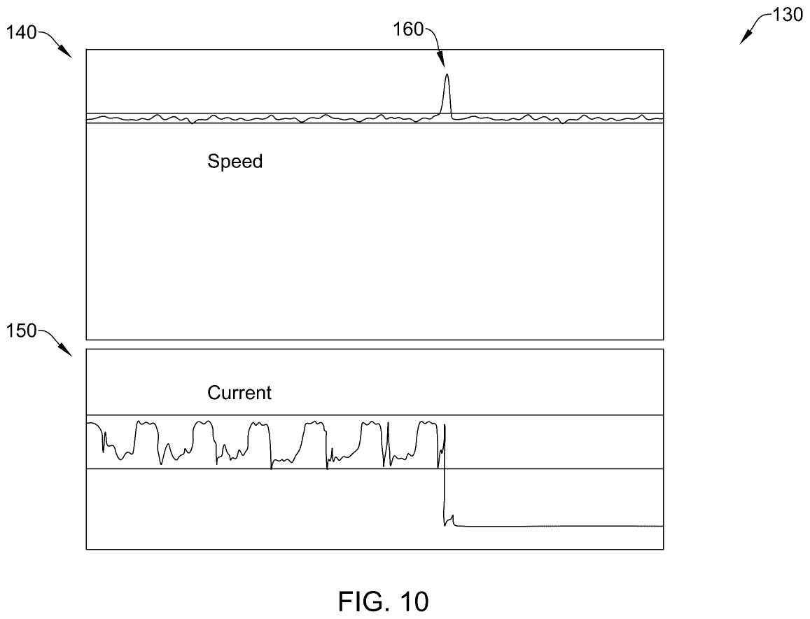

During operation of the blood pump 50 it is desirable to maintain magnetic coupling between the drive magnet 76 and the driven magnet 78. In some instances, the controller may be able to sense the magnetic drive assembly reaching a maximum torque, at which point the controller may be configured to automatically lower the speed of the motor, lower the current to the motor, etc. However, if the maximum torque is exceeded, a magnetic decoupling event will occur in which the magnetic coupling between the drive magnet 76 and the driven magnet 78 is broken. FIG. 10 shows a group of charts comparing time histories of motor speed against applied electrical current in accordance with examples described herein. As shown, a magnetic decoupling event 160 may occur when motor speed spikes as shown in top chart 140 and identified as a magnetic decoupling event 160. As shown in the bottom chart 150 accompanying the top chart 140, the magnetic decoupling event 160 is also indicated by an immediate and rapid drop in electrical current to the motor 72 which may be sensed by the controller (not shown), in which electrical current drops to a near-zero value (as the applied torque rapidly decreases) and may do so instantaneously and in coordination with a rapid uptick in motor speed as shown in the top chart 140.

Magnetic decoupling between the drive magnet (the first dipole magnet) and the driven magnet (the second dipole magnet) may generally occur when the angle between respective north poles of the dipole magnets is less than 90 degrees (e.g., the angle between the north pole of the first dipole magnet exceeds 90 degrees from the south pole of the second dipole magnet), as this angle has been found to be the point of maximum torque that a magnetic coupling between dipole magnets may be sustained. If the impeller system requires more torque to drive than what is allowed by the maximum torque condition, the magnetization directions may exceed an angle of 90 degrees and the magnets will break free from their magnetic coupling. This typically results in a scenario where the drive magnet 76 (which is attached to and rotationally driven by the motor) may continue spinning with the motor 72, but the driven magnet 78 (which is attached to the impeller 67) may magnetically decouple from the drive magnet 76, causing the driven magnet 78 to stop spinning as drag forces from the blood (since the drive magnet is decoupled) act to slow down the driven magnet 78 by impeding the angular velocity and spin rate of the driven magnet 78. In these conditions, the drive magnet 76 (the first dipole magnet) and the driven magnet 78 (the second dipole magnet) cannot be recoupled unless the motor RPM is lowered significantly or stopped to greatly slow or stop rotation of the drive magnet 76.

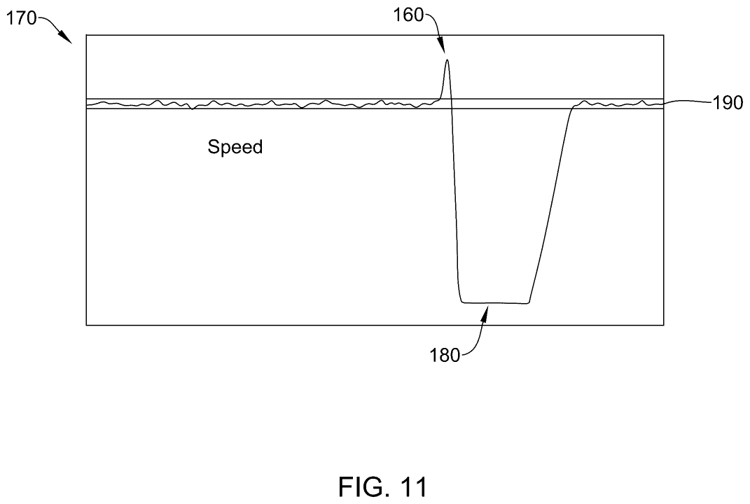

In some embodiments, the controller, that is providing electrical current to the motor 72, may be configured to automatically sense a magnetic decoupling event if an angle between the north pole of the driven magnet 78 and the south pole of the driving magnet 76 exceeds 90 degrees. In other words, the controller may be configured to automatically sense a rapid, momentary spike in the speed and/or acceleration in the motor, and/or subsequent decline in the speed and/or rapid deceleration of the motor, such as an event shown in the chart 170 of FIG. 11. As further shown in FIG. 10, in some instances, the controller may be configured to automatically sense the magnetic decoupling event 160 (such as by sensing a rapid decline in electrical current supplied to the motor, as shown in the bottom chart 150 of FIG. 10), to determine the occurrence of a magnetic decoupling event between the drive magnet 76 and the driven magnet 78. In some instances, the controller may issue an alarm or an alert, such as a visual alarm or alert to a display of the controller and/or an audible alarm or alert, that alerts a user of the device to an automatically sensed magnetic decoupling event.

Upon automatically sensing a magnetic decoupling event, the controller may be configured to automatically reduce the speed of the motor 72 to a baseline speed 180 and/or stop the motor 72 (e.g., zero RPMs) to permit magnetic recoupling between respective poles of the driven magnet 78 and the drive magnet 76, such as shown in FIG. 11. In some instances, the controller may completely stop the motor 72 and thereafter re-start the motor and resume operation of the blood pump at a desired speed (the resumed speed 190) after magnetic recoupling has occurred. In some instances, the controller may re-start the motor and increase the speed to a pre-set speed saved in the controller, an operating speed of the motor just prior to the decoupling event, and/or an operating speed at a lower speed than the operating speed of the motor just prior to the decoupling event. In some instances, the lower speed may be proportional to or a percentage of the operating speed of the motor just prior to the decoupling event, such as 90% or less, 80% or less, 60% or less, or 50% or less than the operating speed of the motor just prior to the decoupling event. In other instances, the lower speed may be a fixed or constant value offset, such as 1,000 RPM less than, 2,000 RPM, less than, 3,000 RPM less than, 4,000 RPM less than, or 5,000 RPM less than the operating speed of the motor just prior to the decoupling event. In other instances, the lower speed may be another speed that the motor resumes operation at. Thus, the controller may permit magnetic recoupling between respective poles of the drive magnet 76 (e.g., the first dipole magnet) and the driven magnet 78 (e.g., the second dipole magnet) without user intervention.

In other instances, if the controller senses the magnetic drive assembly is nearing maximum torque (e.g., approaching a magnetic decoupling event) the controller may be configured to automatically reduce the speed of the motor 72 to a lower speed level (e.g., the next lower speed level) and/or lock out or prevent the user from selecting any higher motor speed levels. In some instances, the controller may limit the electrical current supplied to the motor, such as limit the electrical current supplied to the motor to a percentage (less than 100%), such as 95% or less, or 90% or less, of the current that represents the maximum torque attainable by the magnetic drive assembly prior to a decoupling event.

Any of the magnets, magnetic assemblies, and magnetic couplings described herein may be machined and/or manufactured to any feasible size for implementation within a patient and/or subject, and/or for any experimental use. The diametric dipole magnets, dipole magnets, diametric magnets, and/or other magnets disclosed herein may be machined and/or manufactured to different, alternative and/or differing dimensions (e.g., lengths, widths, thicknesses, diameters, heights, circumferences, etc.). In non-limiting examples, any of the diametric dipole magnets, dipole magnets, diametric magnets and/or other magnets disclosed herein may be provided with a diameter within the range of about 2 mm to about 8 mm. In other non-limiting examples, any of the diametric dipole magnets, dipole magnets, diametric magnets and/or other magnets disclosed herein may be provided with a length within the range of about 1 mm to about 5 mm. However, other ranges and values for the dimensions of any of the diametric dipole magnets, dipole magnets, diametric magnets and/or other magnets disclosed herein are also contemplated.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A percutaneous circulatory support device, comprising:
a housing comprising a blood inlet and a blood outlet;
an impeller disposed within the housing, the impeller configured to rotate relative to the housing to cause blood to flow through the housing from the blood inlet to the blood outlet;
a motor operably coupled to the impeller, the motor configured to rotate the impeller relative to the housing;
an impeller shaft coupled to the impeller and configured to rotate with the impeller;
a driving magnet assembly coupled to the motor, wherein the motor is configured to rotate the driving magnet assembly; and
a driven magnet assembly coupled to at least one of the impeller shaft and the impeller, the driven magnet magnetically coupled to the driving magnet assembly, wherein rotation of the driving magnet assembly causes rotation of the driven magnet assembly and the impeller;
wherein the driving magnet assembly includes a first dipole magnet having a single north pole and a single south pole, and the driven magnet assembly includes a second dipole magnet having a single north pole and a single south pole.

2. The device of claim 1, wherein the driving magnet assembly applies a torque upon the driven magnet assembly during operation of the motor.

3. The device of claim 1, wherein the driving magnet assembly applies a torque upon the driven magnet assembly during rotation of the impeller shaft.

4. The device of claim 1, further comprising a yoke attached to one or more of the first dipole magnet and the second dipole magnet.

5. The device of claim 1, the device comprising a controller operably connected thereto, and wherein:
the controller is configured to automatically sense a magnetic decoupling event if an angle between the north pole of the first dipole magnet and the south pole of the second dipole magnet exceeds 90 degrees.

6. The device of claim 5, wherein the controller is configured to:
issue an alarm or an alert that alerts a user of the device to the sensed magnetic decoupling event.

7. The device of claim 5, wherein the controller is configured to:
automatically reduce the speed of the motor to permit magnetic recoupling between respective poles of the first dipole magnet and the second dipole magnet upon sensing a magnetic decoupling event.

8. The device of claim 7, wherein the controller is configured to:
permit magnetic recoupling between respective poles of the first dipole magnet and the second dipole magnet without user intervention; and
thereafter, automatically increase the speed of the motor.

9. A percutaneous blood pump, comprising:
an impeller housing including an impeller;
a first magnet assembly coupled to the motor such that the motor is configured to rotate the first magnet assembly;
a second magnet assembly coupled to the impeller and configured to rotate with the impeller; and
a wall disposed between the first magnet assembly and the second magnet assembly;
wherein the first magnet assembly is disposed proximal of the wall within the motor housing and remote from the impeller housing;
wherein the second magnet assembly is disposed distal of the wall within the impeller housing and remote from the motor housing;
wherein the first magnet assembly includes a first diametric dipole magnet including a single north pole and a single south pole, and the second magnet assembly includes a second diametric dipole magnet including a single north pole and a single south pole;
wherein the north pole of the first diametric dipole magnet is circumferentially aligned with the south pole of the second diametric dipole magnet at rest.

10. The blood pump of claim 9, wherein the first diametric dipole magnet applies a torque upon the second diametric dipole magnet during operation of the motor.

11. The blood pump of claim 9, wherein the north pole of the first diametric dipole magnet is circumferentially offset from the south pole of the second diametric dipole magnet by an angle of 10 degrees to 85 degrees during normal operation of the motor.

12. The blood pump of claim 9, wherein the first diametric dipole magnet and the second diametric dipole magnet are separated by a distance within a range of about 0.5 mm to about 1.0 mm.

13. The blood pump of claim 9, wherein the wall allows the passage of magnetic forces between the first magnet assembly and the second magnet assembly.

14. The blood pump of claim 9, the blood pump comprising a controller operably connected thereto, wherein:
the controller is configured to automatically sense a magnetic decoupling event between the first diametric dipole magnet and the second diametric dipole magnet if the angle between the north pole of the first diametric dipole magnet and the south pole of the second diametric dipole magnet exceeds 90 degrees.

15. The blood pump of claim 14, wherein the controller is configured to:
issue an alarm or an alert that alerts a user of the device to an automatically sensed magnetic decoupling event.

16. The blood pump of claim 14, wherein the controller is configured to:
automatically reduce the speed of the motor or stop the motor to permit magnetic recoupling between the first diametric dipole magnet and the second diametric dipole magnet upon automatically sensing the magnetic decoupling event.

17. A percutaneous circulatory support device, comprising:
a controller;
a housing comprising a blood inlet and a blood outlet;
an impeller disposed within the housing, the impeller configured to rotate relative to the housing to cause blood to flow through the housing from the blood inlet to the blood outlet;
a motor operably coupled to the impeller, the controller configured to supply electrical current to the motor to rotate the impeller relative to the housing;
a drive shaft coupled to the impeller and configured to rotate with the impeller;
a drive magnet coupled to the motor, wherein the motor is configured to rotate the drive magnet;
a driven magnet coupled to at least one of the drive shaft and the impeller, the driven magnet magnetically coupled to the drive magnet, wherein rotation of the drive magnet causes rotation of the driven magnet and the impeller;
wherein the drive magnet is a dipole magnet having a single north pole and a single south pole, and the driven magnet is a dipole magnet having a single north pole and a single south pole.

18. The device of claim 17, wherein the controller is configured to automatically sense a magnetic decoupling event if an angle between the north pole of the driven magnet and the south pole of the driving magnet exceeds 90 degrees.

19. The device of claim 18, wherein the controller is configured to:
automatically reduce the speed of the motor and/or stop the motor to permit magnetic recoupling between respective poles of the driven magnet and the drive magnet upon automatically sensing the magnetic decoupling event.

20. The device of claim 19, wherein the controller is configured to:
permit magnetic recoupling between respective poles of the driven magnet and the drive magnet without user intervention; and
thereafter, automatically increase the speed of the motor.
